(19) United States
(12) Patent Application Publication
(10) Pub. No.: US 2008/0176213 A1
(43) Pub. Date: Jul. 24, 2008

(54) CONTROL OF CHEMICAL MODIFICATION

(75) Inventors: John Matthew Mauro, Eugene, OR (US); Thomas Harry Steinberg, Eugene, OR (US); Lawrence I. Greenfield, Eugene, OR (US); Louis Leong, Junction City, OR (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/057,258

(22) Filed: Mar. 27, 2008

Related U.S. Application Data
(63) Continuation of application No. 11/470,525, filed on Sep. 6, 2006.
(60) Provisional application No. 60/714,992, filed on Sep. 8, 2005.

(51) Int. Cl. C12Q 1/00; C12N 5/00; C12N 5/02; C12N 1/38

(52) U.S. Cl. 435/4; 435/395; 435/396; 435/397; 435/398; 435/401; 435/403; 435/244

(57) ABSTRACT

Provided is a method for controlling the degree of labeling (DOL) of a carrier molecule or solid support by the addition of a reactive label competitor to the labeling reaction. When the reactive label competitor is added to the labeling solution the competitor competes with the carrier molecule or solid support for the label, reducing the number of labels available to conjugates to the carrier molecule or solid support. This provides for a facile method that predictably alters the DOL of a carrier molecule or solid support.

### CROSS REFERENCE TO RELATED APPLICATION

This is a continuation application of U.S. Ser. No. 11/470,525, filed Sep. 6, 2006 and claims priority to U.S. Ser. No. 60/714,922, filed Sep. 6, 2005, which disclosures are herein incorporated by reference.

### FIELD OF THE INVENTION

The present invention relates to controlling the degree of labeling (DOL) on a carrier molecule or solid support. The invention has applications in the fields of cell biology, in vivo imaging, pathology, neurology, immunology, proteomics and biosensing.

### BACKGROUND OF THE INVENTION

Control of the nature and extent of reaction of two or more chemically reactive components can be achieved by various means that alter reaction kinetics and thermodynamics. Changing reaction volumes and/or reactant concentrations are well-known ways to affect reaction rates. Decreasing the concentration of one or more of the reactants generally has the effect of decreasing the reaction rate, thus reducing the total amount of product obtained during a given period of time. In the case of an excess of a labeling agent reacting with a multivalent receptor, carrier molecule, or a surface having many chemical groups available for derivatization, one way to slow or control the reaction process is to add a reactive competitor for one or more of the initially-present reactants, resulting in a net decrease in the concentration of one or both of the reactants. Examples can be found in photoaffinity labeling experiments, in which scavenger molecules are often included to react with and inactivate photoactivated molecular species that diffuse away from their receptor binding sites while still reactive, thus greatly increasing the apparent selective labeling of the binding site of the photoaffinity probe [Samson M, Osty J, Blondeau JP. Endocrinology. 1993 June; 132(6):2470-6.].

Complete quenching of a reaction is possible by adding a large excess of a competitor at some stage of a reaction, and this is done to effectively stop or quench chemical reactions in many cases. By addition of a smaller controlled amount of a competitor, however, a reaction can be kinetically slowed and controlled and not totally quenched. In certain cases, this competition approach will be preferable to alternative ways of controlling reaction rates, e.g., changing the overall reaction volume at constant mass of reactants or changing the concentration of reactants. An example would be when a subsequent processing step, for instance purification of a reaction product, would be rendered less effective or efficient by significant changes in reaction volume or reactant concentrations. In this case, controlling the kinetics of the reaction by addition of a competitor, for example by addition of a very small volume of concentrated liquid competitor to a large reaction volume could slow and control the reaction while having an insignificant effect on the subsequent volume or concentration-dependent purification step. An example of a purification method that is strongly dependent upon the final reaction volume is gel permeation chromatography [Male CA. Methods Med. Res. 1970; 12:221-41.], where there exists a maximum sample application volume, which when exceeded, the purification process is much less effective or impossible to carry out. In such a case, control of the reaction rate by addition of a very small volume of concentrated competitor will allow purification by gel permeation chromatography to proceed without process changes.

Molecular affinity-based detection depends on both the selectivity of targeting agents for their chosen target sites and on the observation of a signaling center associated with the targeting agent. Retention of selectivity and reactivity of the targeted agent upon its derivatization with a signaling center is critical for successful detection. Among the wide variety of known specific targeting agents are natural antibodies and unnatural fragments of antibodies, a wide range of proteins and peptides, polymerized nucleic acids, polymerized carbohydrates, and templated surfaces. Effective use of an antibody labeled with one or more fluorescent signaling groups in various applications, for example, generally depends upon retention of the physical integrity and chemical selectivity of the antibody after derivatization with the fluorescent groups. Furthermore, the photochemistry and photophysics of the fluorescent signaling groups linked to their performance in a given application often depends upon the total number of groups attached to the antibody [Berlier, J E et al. J. Histochem. Cytochem. 2003; 51(12): 1699-1712.].

For example, a specific molecular imaging probe for use in vivo must possess pharmacokinetic properties such that it reaches its intended target and remains there sufficiently long to be detectable in living subjects. The probe is subject to most or all of the pharmacokinetic rules and constraints that govern the concentration of drugs in plasma, including absorption, distribution, excretion and other factors in the vascular environment. Rapid excretion, nonspecific trapping/binding, metabolism and delivery barriers must be considered when developing and employing probes [Massoud, T F and Gambhir, S S, 2003 Genes and Development 17: 545-80]. In certain applications, such as where the intended use of an antibody is as a molecular imaging probe in vivo in optical-based imaging, the number of fluorescent groups attached to an antibody may be a critical factor in determining its biodistribution, pharmacokinetics, and serum clearance rate. A typical labeling reaction using 0.1 mg of an antibody and 10 to 20 micrograms of an amine-reactive fluorescent dye derivative might produce a product with an average ratio of fluorescent dye to protein (the Degree of Labeling or DOL) of 6-8. It is expected that for some antibodies and some dyes, this DOL will be inappropriate for this application. For example, the antibody may lose stability or selectivity, the dyes themselves may emit less fluorescent light due to self-quenching [Berlier, supra], or the biodistribution, pharmacokinetics, or clearance rates in vivo may be adversely affected at this DOL [Wu, A M et al. Proc. Natl. Acad. Sci. USA 2000; 97(15): 8495-8500].

The ability to alter the DOL in a labeling reaction can be achieved by various means that are relatively predictable by known chemical theory. Changes of concentration or relative concentrations of the reacting species can be made to allow systematic variation of the DOL of the final product. Changes in reaction conditions, such as solution volume, pH, buffer composition, ionic strength, temperature or reaction time can also be used to modulate or control the DOL of the final product. In some cases, however, using alterations in these parameters effectively to control the DOL is subject to considerable trial and error, depending especially on the chemical nature of the reacting species, and can be problematical when the exact chemical behavior of either or both of the reactive species is not completely known or not readily predictable. There may also be cases where employing trial and error to achieve optimum results may eventually be successful, but where the cost or limited availability, for example, of one or more of the reactants is prohibitive for this approach. Existing methods for modulation of DOL in general depend upon alteration of the concentration of the carrier molecule or, more often, alteration of the concentration of the reactive labeling species. Commercial kits exist that employ these methods, for example, the Fluorotag™ FITC Conjugation Kit (Sigma FITC-1). In this kit, small scale conjugations are performed at three different ratios of FITC to protein. Based on the molecular ratio that gives the most satisfactory result, a larger-scale procedure can be performed to optimally label the protein. In the recommended small scale pilot experiments, a non-optimal aspect of the method described in the kit involves dissolving the reactive label in buffer (to allow 20:1 dye:protein in final reaction), making dilutions of the reactive dye to achieve 10:1 and 5:1 dye:protein ratios, then adding these dilutions drop wise to a constant amount of antibody solution, all within 5 minutes of initial dilution of the reactive FITC dye. In sum, the method depends on trial and error and is operationally difficult and cumbersome.

The present invention solves this problem by providing a way to alter conditions in a generally predictable manner that is consistent with convergence to a desired DOL with a minimum of trial and error. It is also simpler to implement, especially in a pre-made kit format, where there is no need to make variable dilutions of the reactive label to allow alteration of DOL. The present invention provides, for the first time, an easy and effective means for controlling the optimum DOL under simple homogeneous reaction conditions without significantly altering the volumes or concentrations of the initial carrier molecule or reactive label reactants.

### SUMMARY OF THE INVENTION

One embodiment of the present invention provides a method of modulating the amount of reactive label present in a solution, said method comprising:
a) contacting a solution comprising a carrier molecule or solid support with a reactive label to form a labeled carrier molecule or labeled solid support; and
b) contacting the solution with a reactive label competitor to form a labeled competitor;
wherein the amount of reactive label in the solution is attenuated or eliminated after contacting the reactive label with the reactive label competitor.

A preferred embodiment provides a method for controlling the degree of labeling (DOL) of a carrier molecule or solid support by a reactive label. The method provides the steps of:
a) contacting the carrier molecule or solid support with a reactive label to form a labeling solution;
b) contacting the labeling solution with a reactive label competitor to form a controlled labeling solution; and
c) incubating the controlled labeling solution for an appropriate amount of time whereby the degree of labeling of the carrier molecule or solid support is controlled.

When the reactive label competitor is added to the labeling solution the competitor competes with the carrier molecule or solid support for the label, reducing the number of labels available to conjugate to the carrier molecule or solid support, See FIG. 7. This provides for a facile method that predictably alters the DOL of a carrier molecule or solid support.

In one aspect the carrier molecule comprises a amino acid, a peptide, a protein, a polysaccharide, a nucleotide, a nucleoside, an oligonucleotide, a nucleic acid, a hapten, a psoralen, a drug, a hormone, a lipid, a lipid assembly, a synthetic polymer, a polymeric microparticle, a biological cell or a virus. In a further aspect, the carrier molecule comprises an antibody or fragment thereof, an avidin or streptavidin, a biotin, a blood component protein, a dextran, an enzyme, an enzyme inhibitor, a hormone, an IgG binding protein, a fluorescent protein, a growth factor, a lectin, a lipopolysaccharide, a microorganism, a metal binding protein, a metal chelating moiety, a non-biological microparticle, a peptide toxin, a phosphotidylserine-binding protein, a structural protein, a small-molecule drug, or a tyramide.

In another aspect the solid support comprises a microfluidic chip, a silicon chip, a microscope slide, a microplate well, silica gels, polymeric membranes, particles, derivatized plastic films, glass beads, cotton, plastic beads, alumina gels, polysaccharides, polyvinylchloride, polypropylene, polyethylene, nylon, latex bead, magnetic bead, paramagnetic bead, and superparamagnetic bead. In a further aspect the solid support comprises Sepharose™, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose and starch.

In another aspect the reactive label comprises a fluorophore, a phosphorescent dye, a tandem dye, a particle, an electron transfer agent, biotin or a radioisotope. In a further aspect the fluorophore is dansyl, xanthene, naphthalene, coumarin, cyanine, pyrene, or derivatives thereof. In one embodiment the fluorophore has an emission spectra greater than about 600 nm.

The reactive label comprises a reactive group that comprises an acrylamide, an activated ester of a carboxylic acid, a carboxylic ester, an acyl azide, an acyl nitrile, an aldehyde, an alkyl halide, an anhydride, an aniline, an amine, an aryl halide, an azide, an aziridine, a boronate, a diazoalkane, a haloacetamide, a haloalkyl, a halotriazine, a hydrazine, an imido ester, an isocyanate, an isothiocyanate, a maleimide, a phosphoramidite, a reactive platinum complex, a silyl halide, a sulfonyl halide, or a thiol. In one aspect the reactive group comprises a carboxylic acid, succinimidyl ester of a carboxylic acid, hydrazide, amine or a maleimide.

The reactive label competitor and carrier molecule or solid support, independently comprise an amino group or thiol group. For a particular labeling reaction the reactive label competitor and carrier molecule or solid support will both comprise an amine group, a thiol group, or another type of reactive group.

In one aspect the reactive label competitor comprises an amino group. In a further aspect, the reactive label competitor comprises α-amino acids, β-amino acids, amino alcohols, ε-amino acids, primary amine containing compounds or reactive secondary amine-containing compounds. In yet a further embodiment, the reactive label competitor comprises D-lysine, L-lysine, D,L-lysine, ethanoloamine, 5-amino caproic acid, or ammonia (NH₃). In a particularly preferred embodiment the reactive label competitor is L-Lysine Hydrochloride.

In another aspect the reactive label competitor comprises a thiol group. In this instance the reactive label competitor comprises α-mercapto acids, β-mercapto acids, mercapto alcohols, ε-mercapto acids, primary mercaptan compounds or reactive secondary mercaptan compounds. In one aspect, the reactive label competitor comprises D-cysteine, L-cysteine, D,L-cysteine, mercaptoethanol, 5-mercapto caproic acid, or H₂S

Another aspect further comprises a step of separating labeled competitor from the labeled carrier molecule or labeled solid support. More particular still, the step of separating comprises column chromatography.

Provided in another embodiment is a kit for controlling the degree of labeling (DOL) of a carrier molecule or solid support, wherein the kit comprises:
a) a reactive label;
b) a reactive label competitor; and
c) instructions for performing a method resulting in the controlled degree of labeling of the carrier molecule or solid support.

Provided in another embodiment is a kit for controlling the degree of labeling (DOL) of a carrier molecule or solid support, wherein the kit comprises:
a) carrier molecule or solid support;
b) a reactive label;
c) a reactive label competitor; and
d) instructions for performing a method resulting in the controlled degree of labeling of the carrier molecule or solid support.

In a more particular embodiment the kit also comprises at least one additional element selected from the group consisting of:
e) a buffer;
f) a salt;
g) a purification column;
h) a purification resin; and
i) a syringe and syringe filters.

In a more particular embodiment the kit comprises at least two, three, four or all of the additional elements. In another more particular embodiment the buffer is phosphate buffered saline (PBS). In another more particular embodiment the salt is sodium bicarbonate.

Additional embodiments are described in the detailed description of the invention.

### BRIEF DESCRIPTION OF THE FIGURES

FIG. 1: Shows that addition of appropriate amounts of primary amine in the form of free lysine can result in modulation of the degree of labeling (DOL) of proteins, as demonstrated with Goat anti-mouse IgG (GAM), using Alexa Fluor 647 succinimidyl ester (SE) dye. See, Example 1.

FIG. 2: Shows that free lysine, present in different concentrations, can control the DOL in a predictable way for bovine serum albumin (BSA), Goat anti-mouse (GAR) IgG, Streptavidin, and Transferrin when conjugated to Alexa Fluor 647 SE dye and Alexa Fluor 680 SE dye.

FIG. 3: Shows the labeling modulation of Alexa Fluor 647 SE dye and Alexa Fluor 680 SE dye conjugated to Goat anti-rabbit (GAR) antibody by the addition of different concentrations of lysine. The addition of 0.3 mM lysine results in about a 40% reduction in labeling of the IgG with dye and the addition of 1 mM lysine results in about a 70% reduction in labeling of the IgG with reactive dye. Results were about the same for a 60 minute incubation period at room temperature or for about a 20 hour incubation period on ice. Error bars are one standard deviation. See Example 3.

FIG. 4: Shows the labeling modulation of Alexa Fluor 647 SE dye and Alexa Fluor 680 SE dye conjugated to F(ab′)₂ (FIGS. 4A and B), Fab′ (FIGS. 4C and D) and Transferrin (FIGS. 4E and F) by the addition of different concentrations of lysine. See, Example 3.

FIG. 5: Shows the labeling modulation of Alexa Fluor 647 SE dye (FIG. 5A) and Alexa Fluor 680 SE dye (FIG. 5B) conjugated to different concentrations (3 mg/ml, 1 mg/ml and 0.3 mg/ml) of Goat anti-rabbit (GAR) antibody in the presence of different concentrations of lysine. See, Example 3.

FIG. 6: Shows the labeling modulation of Alexa Fluor 750 SE dye conjugated to Goat anti-rabbit (GAR) antibody by the addition of different concentrations of lysine incubated for 60 minutes are room temperature or for about 20 hours on ice.

FIG. 7: Shows a schematic illustration of facile alteration of DOL for labeling carrier molecule A with label B by addition to the reaction of reactive group C.

FIG. 8: Shows concentration of reactive species as a function of pH. The protein concentration is 1 mg/mL. It is assumed that the molecule weight of the protein is 150,000, there are 10 available lysines per protein and the pKa of the reactive lysines are 9.8. The relative concentrations of the reactive amines and hydroxide ion concentrations track each other until close to the pKₐ of lysine. At a pH close to lysine pKₐ, the concentration of reactive lysines (deprotonated amines) begin to reach saturation at the concentration of amines present in the reaction.

FIG. 9: Shows the reactive species as a function of antibody concentration and the contribution of overall reactive species contributed by the antibody as a function of protein concentration. It is assumed that the pH of the reaction is 8.0, the molecular weight of the protein is 150,000, there are 10 available lysines per protein and the pKa of the reactive lysines are 9.8. The fractional concentration of reactive amines was calculated using Eq. 2.

FIG. 10: Shows the effect of protein concentration on the amount of degree of labeling of antibody (three independent experiments).

FIG. 11: Shows the relationship between the fraction of reactive protein groups and the degree of protein derivatization. (Result of three independent experiments). The fraction of total deprotonated antibody amine was calculated using Eq. 2. The solid dark straight line is the theoretical data fit.

FIG. 12: Shows the predicted effect of addition of an attenuator species (lysine), converting the relationship between fraction of reactive species contributed by the protein to a linear function of protein concentration.

FIG. 13: Shows the concentration of deprotonated amine and hydroxide ion concentration as a function of pH

FIG. 14: Shows the effect of reactive label competitor addition (1.4 mM Lysine) on protein concentration-dependent antibody derivatization. (Data represents two separate experiments).

FIG. 15: Shows the effect of reactive label competitor addition on the degree of derivatization of BSA.

FIG. 16: Shows the effect of reactive label competitor addition on degree of derivatization of a polyclonal goat anti-rabbit antibody.

FIG. 17: Shows the effect of reactive label competitor addition on antibody derivatization using NHS-ester forms of AlexaFluor 647 (Dye 1), AlexaFluor 680 (Dye 2) and AlexaFluor 750 (Dye 3).

### DETAILED DESCRIPTION OF THE INVENTION

### Introduction

The present invention provides methods for controlling the degree of labeling of a carrier molecule or solid support without significantly altering protein concentrations, label concentrations, reaction volume or reaction time. The present method is accomplished by adding a “reactive label competitor” to the labeling reaction.

### DEFINITIONS

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a carrier molecule” includes a plurality of molecules and reference to “a label” includes a plurality of labels and the like.

Unless defined otherwise, all technical and scientific terms used herein have the same meaning as commonly understood by one of ordinary skill in the art to which this invention is related. The following terms are defined for purposes of the invention as described herein.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “Antibody” as used herein refers to a protein of the immunoglobulin (Ig) superfamily that binds noncovalently to certain substances (e.g. antigens and immunogens) to form an antibody-antigen complex, including but not limited to antibodies produced by hybridoma cell lines, by immunization to elicit a polyclonal antibody response, by chemical synthesis, and by recombinant host cells that have been transformed with an expression vector that encodes the antibody. In humans, the immunoglobulin antibodies are classified as IgA, IgD, IgE, IgG, and IgM and members of each class are said to have the same isotype. Human IgA and IgG isotypes are further subdivided into subtypes IgA₁, and IgA₂, and IgG₁, IgG₂, IgG₃, and IgG₄. Mice have generally the same isotypes as humans, but the IgG isotype is subdivided into IgG₁, IgG₂a, IgG₂b, and IgG₃ subtypes. Thus, it will be understood that the term “antibody” as used herein includes within its scope (a) any of the various classes or sub-classes of immunoglobulin, e.g., IgG, IgM, IgE derived from any of the animals conventionally used and (b) polyclonal and monoclonal antibodies, such as murine, chimeric, or humanized antibodies. Antibody molecules have regions of amino acid sequences that can act as an antigenic determinant, e.g. the Fc region, the kappa light chain, the lambda light chain, the hinge region, etc. An antibody that is generated against a selected region is designated anti-[region], e.g. anti-Fc, anti-kappa light chain, anti-lambda light chain, etc. An antibody is typically generated against an antigen by immunizing an organism with a macromolecule to initiate lymphocyte activation to express the immunoglobulin protein. The term antibody, as used herein, also covers any polypeptide or protein having a binding domain that is, or is homologous to, an antibody binding domain, including, without limitation, single-chain Fv molecules (scFv), wherein a VH domain and a VL domain are linked by a peptide linker that allows the two domains to associate to form an antigen binding site (Bird et al., Science 242, 423 (1988) and Huston et al., Proc. Natl. Acad. Sci. USA 85, 5879 (1988)). These can be derived from natural sources, or they may be partly or wholly synthetically produced.

The term “Antibody fragments” as used herein refers to fragments of antibodies that retain the principal selective binding characteristics of the whole antibody. Particular fragments are well-known in the art, for example, Fab, Fab′, and F(ab′)₂, which are obtained by digestion with various proteases and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1 domains; (ii) the dAb fragment (Ward, et al., Nature 341, 544 (1989)), which consists of a VH domain; (iii) isolated CDR regions; and (iv) single-chain Fv molecules (scFv) described above. In addition, arbitrary fragments can be made using recombinant technology that retains antigen-recognition characteristics.

The term “amino” or “amine group” refers to the group —NR′R″ (or N⁺RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N⁺RR′R″ and its biologically compatible anionic counterions.

The term “aqueous solution” as used herein refers to a solution that is predominantly water and retains the solution characteristics of water. Where the aqueous solution contains solvents in addition to water, water is typically the predominant solvent.

The term “carrier molecule” as used herein refers to a biological or a non-biological compound that is covalently conjugated to a reactive label. Such compounds include, but are not limited to, an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof.

The term “degree of labeling” or “DOL” as used herein refers to the number of labels that are covalently conjugated to an individual carrier molecule or solid support. Typically the DOL of a carrier molecule or solid support varies over a 10-fold or greater range of covalently bonded dye to carrier molecule or solid support in the final modified, or labeled, carrier molecule or solid support. The term “degree of substitution” or “DOS” is used interchangeably with DOL.

The term “detectable response” as used herein refers to a change in or an occurrence of, a signal that is directly or indirectly detectable either by observation or by instrumentation and the presence or magnitude of which is a function of the presence of a target metal ion in the test sample. Typically, the detectable response is an optical response resulting in a change in the wavelength distribution patterns or intensity of absorbance or fluorescence or a change in light scatter, fluorescence quantum yield, fluorescence lifetime, fluorescence polarization, a shift in excitation or emission wavelength or a combination of the above parameters. The detectable change in a given spectral property is generally an increase or a decrease. However, spectral changes that result in an enhancement of fluorescence intensity and/or a shift in the wavelength of fluorescence emission or excitation are also useful. The change in fluorescence on ion binding is usually due to conformational or electronic changes in the indicator that may occur in either the excited or ground state of the fluorophore, due to changes in electron density at the ion binding site, due to quenching of fluorescence by the bound target metal ion, or due to any combination of these or other effects. Alternatively, the detectable response is an occurrence of a signal wherein the fluorophore is inherently fluorescent and does not produce a change in signal upon binding to a metal ion or biological compound.

The term “fluorophore” as used herein refers to a composition that is inherently fluorescent or demonstrates a change in fluorescence upon binding to a biological compound or metal ion, or metabolism by an enzyme, i.e., fluorogenic. Fluorophores may be substituted to alter the solubility, spectral properties or physical properties of the fluorophore. Numerous fluorophores are known to those skilled in the art and include, but are not limited to coumarin, acridine, furan, dansyl, cyanine, pyrene, naphthalene, benzofurans, quinolines, quinazolinones, indoles, benzazoles, borapolyazaindacenes, oxazine and xanthenes, with the latter including fluoresceins, rhodamines, rosamine and rhodols as well as other fluorophores described in RICHARD P. HAUGLAND, MOLECULAR PROBES HANDBOOK OF FLUORESCENT PROBES AND RESEARCH CHEMICALS (9^{th} edition, including the CD-ROM, September 2002). The fluorophore moiety may be substituted by substituents that enhance solubility, live cell permeability and alter spectra absorption and emission.

The term “kit” as used refers to a packaged set of related components, typically one or more compounds or compositions.

The term “Label” as used herein refers to a chemical moiety or protein that retains its native properties (e.g. spectral properties, conformation and activity) when conjugated to a carrier molecule or solid support. Illustrative labels include labels that can be directly observed or measured or indirectly observed or measured. Such labels include, but are not limited to, pigments, dyes or other chromogens that can be visually observed or measured with a spectrophotometer; spin labels that can be measured with a spin label analyzer; and fluorescent moieties, where the output signal is generated by the excitation of a suitable molecular adduct and that can be visualized by excitation with light that is absorbed by the dye or can be measured with standard fluorometers or imaging systems, for example. The label can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The label may also take the form of a chemical or biochemical, or an inert particle, including but not limited to colloidal gold, microspheres, nanocrystals, (see, e.g., Beverloo, et al., Anal. Biochem. 203, 326-34 (1992)). The term label can also refer to a “tag” or hapten that can bind selectively to a labeled molecule such that the labeled molecule, when added subsequently, is used to generate a detectable signal. For instance, one can use biotin, iminobiotin or desthiobiotin as a tag and then use an avidin or streptavidin conjugate of horseradish peroxidase (HRP) to bind to the tag, and then use a chromogenic substrate (e.g., tetramethylbenzidine) or a fluorogenic substrate such as Amplex Red or Amplex Gold (Molecular Probes, Inc.) to detect the presence of HRP. In a similar fashion, the tag can be a hapten or antigen (e.g., digoxigenin), and an enzymatically, fluorescently, or radioactively labeled antibody can be used to bind to the tag. Numerous labels are known by those of skill in the art and include, but are not limited to, particles, fluorescent dyes, haptens, enzymes and their chromogenic, fluorogenic, and chemiluminescent substrates, and other labels that are described in the MOLECULAR PROBES HANDBOOK OF FLUORESCENT PROBES AND RESEARCH CHEMICALS by Richard P. Haugland, 6^{th} Ed., (1996), and its subsequent 7^{th} edition and 8^{th} edition updates issued on CD Rom in November 1999 and May 2001, respectively, the contents of which are incorporated by reference, and in other published sources.

The terms “protein” and “polypeptide” are used herein in a generic sense to include polymers of amino acid residues of any length. The term “peptide” as used herein refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in Chemistry and Biochemistry of Amino Acids, Peptides and Proteins, B. Weinstein, eds., Marcel Dekker, New York, p. 267 (1983).

The term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as carboxylic acid or succinimidyl ester, on the compounds of the present invention that is capable of chemically reacting with a functional group on a different compound to form a covalent linkage. Reactive groups generally include nucleophiles, electrophiles and photoactivatable groups.

Exemplary reactive groups include, but are not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like.

Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “reactive label” as used herein refers to a label, as disclosed above, that comprises a reactive group, as disclosed above. The reactive label, under appropriate conditions, forms a covalent bond with a carrier molecule, solid support or reactive label competitor.

The term “reactive label competitor” as used herein refers to a reactive label that contains a nucleophile or electrophile that reacts with the reactive label and will compete for the label, controlling the number of labels available to react with the carrier molecule or solid support. This may be a small molecule or a macromolecule. Reactive label competitors, include, but are not limited to, α-amino acids, β-amino acids (D-lysine, L-lysine, D,L-lysine), amino alcohols (ethanoloamine), ε-amino acids (5-amino caproic acid), primary amine containing compounds (ammonia (NH₃)), reactive secondary amine-containing compounds, α-mercapto acids, β-mercapto acids (D-cysteine, L-cysteine, D,L-cysteine), mercapto alcohols (mercaptoethanol), ε-mercapto acids (5-mercapto caproic acid), primary mercaptan compounds (H₂S) or reactive secondary mercaptan compounds.

The term “essentially unaffected” as used herein indicates substantially no change in the desired product as a result of alteration in reaction conditions. For example, in the presence of a reactive label competitor, the amount of labeled carrier molecule or solid support (i.e. product) is essentially unaffected by the concentration of starting material (unlabeled carrier molecule or solid support). The final solution may be significantly affected by varying the starting materials, such as additional impurities or side products, however the desired product will not.

### The Reactants

In general, for ease of understanding the present invention, the reactants (reactive label, carrier molecule, solid support and reactive label competitor) will first be described in detail, followed by the many and varied methods in which the reactants find uses, which is followed by exemplified methods of use.

Provided is a method for controlling the degree of labeling (DOL) of a carrier molecule or solid support by a reactive label. In particular, the concentrations, volumes and incubation times of the reactants (carrier molecule, solid support and reactive label) are not significantly altered. Instead a reactive label competitor is added, in an appropriate concentration, to alter the DOL in a predictable and reproducible manner. This provides, for the first time that we are aware of, a method for easily controlling the DOL to generate conjugated carrier molecules or solid supports with a desired DOL.

The present invention works by providing a chemical species within a reaction mixture that is capable of reacting with one or more of the reactive components normally present in a standard reaction mixture. Thus, instead of using reaction conditions where A+nB=ABₙ, where A, for example, is a carrier molecule or solid support and B is a reactive label capable of reacting selectively at n sites on A under defined reaction conditions, in the present invention another component, C, reactive label competitor, is provided so that a portion of B can react with C as follows: B+C=BC. Adding modulating component C to the reaction therefore lowers the concentration of B available to react with A to give the desired labeled product.

Under conditions where the in situ reaction B+C=BC results in a decrease in concentration of reactive B in solution, the rate of decrease of concentration of reactive B over time is greater than the decrease in reactive B that would occur by spontaneous hydrolysis in aqueous solvent without the presence of modulator compound C. It is therefore possible that control of DOL using this method, in which the concentration of B drops relatively rapidly over a given time period under given solution conditions, could allow labeling of a different population of reactive amines on, for example, the surface of a protein target, than would be achieved by labeling of the same protein using standard labeling techniques. The net result could be an altered distribution of labeled surface amines (lysines), with potentially different properties of the resulting product (e.g. improved quantum yield for labeling with a fluorescent dye or different activity for a labeled enzyme), compared with the product obtained using a standard non-modulated labeling technique.

### Carrier Molecules (Reactant A)

A variety of carrier molecules are useful in the present invention wherein the reactive label is covalent bonded to the carrier molecule during a conjugation reaction. The presence of the reactive label competitor alters, in a predictable way, the number of label molecules conjugated to the carrier molecule. Exemplary carrier molecules include antibodies, antibody fragments, antigens, steroids, vitamins, drugs, haptens, metabolites, toxins, environmental pollutants, amino acids, peptides, proteins, nucleic acids, nucleic acid polymers, carbohydrates, lipids, and polymers. In one aspect the carrier molecule comprises an amino group(s). In another aspect, the carrier molecule comprises a thiol group(s).

In an exemplary embodiment, the carrier molecule comprises an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. In another exemplary embodiment, the carrier molecule is selected from a hapten, a nucleotide, an oligonucleotide, a nucleic acid polymer, a protein, a peptide or a polysaccharide. In a preferred embodiment the carrier molecule is amino acid, a peptide, a protein, a polysaccharide, a nucleoside, a nucleotide, an oligonucleotide, a nucleic acid, a hapten, a psoralen, a drug, a hormone, a lipid, a lipid assembly, a tyramine, a synthetic polymer, a polymeric microparticle, a biological cell, cellular components, an ion chelating moiety, an enzymatic substrate or a virus. In another preferred embodiment, the carrier molecule is an antibody or fragment thereof, an antigen, an avidin or streptavidin, a biotin, a dextran, an IgG binding protein, a fluorescent protein, agarose, and a non-biological microparticle.

The carrier molecule may include a reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for conjugating the reactive label to the carrier molecule. Useful reactive groups are disclosed below and are equally applicable to the carrier molecule reactive functional groups herein.

In an exemplary embodiment, the carrier is an enzymatic substrate selected from an amino acid, peptide, sugar, alcohol, alkanoic acid, 4-guanidinobenzoic acid, nucleic acid, lipid, sulfate, phosphate, —CH₂OCOalkyl and combinations thereof. Enzyme substrates can be cleaved by enzymes selected from the group consisting of peptidase, phosphatase, glycosidase, dealkylase, esterase, guanidinobenzotase, sulfatase, lipase, peroxidase, histone deacetylase, endoglycoceramidase, exonuclease, reductase and endonuclease.

In another exemplary embodiment, the carrier molecule is an amino acid (including those that are protected or are substituted by phosphates, carbohydrates, or C₁ to C₂₂ carboxylic acids), or a polymer of amino acids such as a peptide or protein. In a related embodiment, the carrier molecule contains at least five amino acids, more preferably 5 to 36 amino acids.

Exemplary peptides include, but are not limited to, neuropeptides, cytokines, toxins, protease substrates, and protein kinase substrates. Other exemplary peptides may function as organelle localization peptides, that is, peptides that serve to target the conjugated compound for localization within a particular cellular substructure by cellular transport mechanisms. Preferred protein carrier molecules include enzymes, antibodies, lectins, glycoproteins, histones, albumins, lipoproteins, avidin, streptavidin, protein A, protein G, phycobiliproteins and other fluorescent proteins, hormones, toxins and growth factors. Typically, the protein carrier molecule is an antibody, an antibody fragment, avidin, streptavidin, a toxin, a lectin, or a growth factor.

In another exemplary embodiment, the carrier molecule comprises a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyl linkage (U.S. Pat. No. 4,711,955) or other linkage. In another exemplary embodiment, the nucleotide carrier molecule is a nucleoside or a deoxynucleoside or a dideoxynucleoside.

Exemplary nucleic acid polymer carrier molecules are single- or multi-stranded, natural or synthetic DNA or RNA oligonucleotides, or DNA/RNA hybrids, or incorporating an unusual linker such as morpholine derivatized phosphates (AntiVirals, Inc., Corvallis Oreg.), or peptide nucleic acids such as N-(2-aminoethyl)glycine units, where the nucleic acid contains fewer than 50 nucleotides, more typically fewer than 25 nucleotides.

In another exemplary embodiment, the carrier molecule comprises a carbohydrate or polyol that is typically a polysaccharide, such as dextran, FICOLL, heparin, glycogen, amylopectin, mannan, inulin, starch, agarose and cellulose, or is a polymer such as a poly(ethylene glycol). In a related embodiment, the polysaccharide carrier molecule includes dextran, agarose or FICOLL.

In another exemplary embodiment, the carrier molecule comprises a lipid (typically having 6-25 carbons), including glycolipids, phospholipids, and sphingolipids. Alternatively, the carrier molecule comprises a lipid vesicle, such as a liposome, or is a lipoprotein (see below).

Some lipophilic substituents are useful for facilitating transport of the conjugated dye into cells or cellular organelles.

Alternatively, the carrier molecule is a cell, cellular system(s), cellular fragment, or subcellular particle(s), including virus particles, bacterial particles, virus components, biological cells (such as animal cells, plant cells, bacteria, or yeast), or cellular components. Examples of cellular components that are useful as carrier molecules include lysosomes, endosomes, cytoplasm, nuclei, histones, mitochondria, Golgi apparatus, endoplasmic reticulum and vacuoles.

In another exemplary embodiment, the carrier molecule non-covalently associates with organic or inorganic materials. Exemplary embodiments of the carrier molecule that possess a lipophilic substituent can be used to target lipid assemblies such as biological membranes or liposomes by non-covalent incorporation of the dye compound within the membrane, e.g., for use as probes for membrane structure or for incorporation in liposomes, lipoproteins, films, plastics, lipophilic microspheres or similar materials.

In an exemplary embodiment, the carrier molecule comprises a specific binding pair member wherein the labels are conjugated to a specific binding pair member and used to the formation of the bound pair. Alternatively, the presence of the labeled specific binding pair member indicates the location of the complementary member of that specific binding pair; each specific binding pair member having an area on the surface or in a cavity which specifically binds to, and is complementary with, a particular spatial and polar organization of the other. Exemplary binding pairs are set forth in Table 2.

TABLE 2
Representative Specific Binding Pairs

| antigen | antibody |
|---|---|
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DNA (RNA) | cDNA (cRNA)† |
| 1 hormone | hormone receptor |
| ion | chelator |

*IgG is an immunoglobulin
†cDNA and cRNA are the complementary strands used for hybridization

In a particular aspect the carrier molecule is an antibody fragment, such as, but not limited to, anti-Fc, an anti-Fc isotype, anti-J chain, anti-kappa light chain, anti-lambda light chain, or a single-chain fragment variable protein; or a non-antibody peptide or protein, such as, for example but not limited to, soluble Fc receptor, protein G, protein A, protein L, lectins, or a fragment thereof. In one aspect the carrier molecule is a Fab fragment specific to the Fc portion of the target-binding antibody or to an isotype of the Fc portion of the target-binding antibody (U.S. Ser. No. 10/118,204). The monovalent Fab fragments are typically produced from either murine monoclonal antibodies or polyclonal antibodies generated in a variety of animals, for example but not limited to, rabbit or goat. These fragments can be generated from any isotype such as murine IgM, IgG₁, IgG₂ₐ, IgG_{2b} or IgG₃.

Alternatively, a non-antibody protein or peptide such as protein G, or other suitable proteins, can be used alone or coupled with albumin. Preferred albumins include human and bovine serum albumins or ovalbumin. Protein A, G and L are defined to include those proteins known to one skilled in the art or derivatives thereof that comprise at least one binding domain for IgG, i.e. proteins that have affinity for IgG. These proteins can be modified but do not need to be and are conjugated to a reactive label in the same manner as the other carrier molecules of the invention.

In another aspect the carrier molecule is a whole intact antibody. Antibody is a term of the art denoting the soluble substance or molecule secreted or produced by an animal in response to an antigen, and which has the particular property of combining specifically with the antigen that induced its formation. Antibodies themselves also serve are antigens or immunogens because they are glycoproteins and therefore are used to generate anti-species antibodies. Antibodies, also known as immunoglobulins, are classified into five distinct classes—IgG, IgA, IgM, IgD, and IgE. The basic IgG immunoglobulin structure consists of two identical light polypeptide chains and two identical heavy polypeptide chains (linked together by disulfide bonds).

When IgG is treated with the enzyme papain, a monovalent antigen-binding fragment can be isolated, referred herein to as a Fab fragment. When IgG is treated with pepsin (another proteolytic enzyme), a larger fragment is produced, F(ab′)₂. This fragment can be split in half by treating with a mild reducing buffer that results in the monovalent Fab′ fragment. The Fab′ fragment is slightly larger than the Fab and contains one or more free sulfhydryls from the hinge region (which are not found in the smaller Fab fragment). The term “antibody fragment” is used herein to define the Fab′, F(ab′)₂ and Fab portions of the antibody. It is well known in the art to treat antibody molecules with pepsin and papain in order to produce antibody fragments (Gorevic et al., Methods of Enzyol., 116:3 (1985)).

The monovalent Fab fragments of the present invention are produced from either murine monoclonal antibodies or polyclonal antibodies generated in a variety of animals that have been immunized with a foreign antibody or fragment thereof, U.S. Pat. No. 4,196,265 discloses a method of producing monoclonal antibodies. Typically, secondary antibodies are derived from a polyclonal antibody that has been produced in a rabbit or goat but any animal known to one skilled in the art to produce polyclonal antibodies can be used to generate anti-species antibodies. The term “primary antibody” describes an antibody that binds directly to the antigen as opposed to a “secondary antibody” that binds to a region of the primary antibody. Monoclonal antibodies are equal, and in some cases, preferred over polyclonal antibodies provided that the ligand-binding antibody is compatible with the monoclonal antibodies that are typically produced from murine hybridoma cell lines using methods well known to one skilled in the art.

In one aspect the antibodies are generated against only the Fc region of a foreign antibody. Essentially, the animal is immunized with only the Fc region fragment of a foreign antibody, such as murine. The polyclonal antibodies are collected from subsequent bleeds, digested with an enzyme, pepsin or papain, to produce monovalent fragments. The fragments are then affinity purified on a column comprising whole immunoglobulin protein that the animal was immunized against or just the Fc fragments.

### Solid Supports (Reactant A)

A solid support suitable for use in the present invention is typically substantially insoluble in liquid phases. Solid supports of the current invention are not limited to a specific type of support. Rather, a large number of supports are available and are known to one of ordinary skill in the art. In one aspect the solid support comprises an amino group(s). In another aspect, the solid support comprises a thiol group(s).

Useful solid supports include solid and semi-solid matrixes, such as aerogels and hydrogels, resins, beads, biochips (including thin film coated biochips), microfluidic chips, silicon chips, multi-well plates (also referred to as microtitre plates or microplates), membranes, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

The solid support may include a reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for conjugating the reactive label to the solid support. Useful reactive groups are disclosed below and are equally applicable to the solid support reactive functional groups herein.

A suitable solid phase support can be selected on the basis of desired end use and suitability for various synthetic protocols. For example, where amide bond formation is desirable to attach the labels to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories).

### Labels (Reactant B)

The labels of the present invention confer a detectable signal, directly or indirectly, to the carrier molecule or solid support to which they are conjugated. These labels also comprise a reactive group, as described below, used to form a covalent bond to the carrier molecule or solid support. The terms labels and reactive labels are used interchangeably.

The present labels can be any label known to one skilled in the art. A wide variety of chemically reactive fluorescent dyes that may be suitable for conjugation are already known in the art (RICHARD P. HAUGLAND, MOLECULAR PROBES HANDBOOK OF FLUORESCENT PROBES AND RESEARCH PRODUCTS (2002)). Labels include, without limitation, a fluorophore, a fluorescent protein, a tandem dye (energy transfer pair), a phosphorescent dye, a particle (e.g., semiconductor nanocrystal or resonance light scattering particle), an electron transfer agent, or a hapten (e.g., biotin). Preferably, the label is a fluorophore wherein the DOL of a protein is modulated by a reactive label competitor resulting in a protein conjugated to specified number of fluorophore molecules.

A fluorescent dye or fluorophore of the present invention is any chemical moiety that exhibits an absorption maximum beyond 280 nm. Dyes of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Ser. Nos. 09/968,401; 09/969,853 and 11/150,596 and U.S. Pat. Nos. 6,403,807; 6,348,599; 5,486,616; 5,268,486; 5,569,587; 5,569,766; 5,627,027; 6,664,047 and 6,048,982), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Fluorinated xanthene dyes have been described previously as possessing particularly useful fluorescence properties (Int. Publ. No. WO 97/39064 and U.S. Pat. No. 6,162,931).

Preferred dyes of the invention include dansyl, xanthene, cyanine, borapolyazaindacene, pyrene, naphthalene, coumarin, oxazine and derivatives thereof. Preferred xanthenes are fluorescein, rhodamine and derivatives thereof, naphthalene and dansyl.

In one embodiment the dye has an emission spectra greater than about 600 nm. In a further embodiment the dye or fluorophore has an emission spectra greater than about 620 nm, an emission spectra greater than about 650 nm, an emission spectra great than about 700 nm, an emission spectra greater than about 750 nm, or an emission spectra greater than about 800 nm. In particularly preferred embodiment the dye has an emission spectra greater than about 600 nm wherein the DOL has been modulated resulting in a conjugated protein optimized for in vivo imaging. In one aspect the dye is a cyanine dye. Preferred are those dyes sold under the trade name Alexa Fluor® dye or spectrally similar dyes sold under the trade names Cy® dyes, Atto dyes or Dy® dyes. Preferred Alexa Fluor dyes include Alexa Fluor 647 dyes, Alexa Fluor 660 Dye, Alexa Fluor 680 dye, Alexa Fluor 700 dye and Alexa Fluor 750 dye.

Typically the dye contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art.

In an exemplary embodiment, the dyes are independently substituted by substituents selected from the group consisting of hydrogen, halogen, amino, substituted amino, alkyl, substituted alkyl, aryl, substituted aryl, heteroaryl, substituted heteroaryl, alkoxy, sulfo, reactive group and carrier molecule. In another embodiment, the xanthene dyes of this invention comprise both compounds substituted and unsubstituted on the carbon atom of the central ring of the xanthene by substituents typically found in the xanthene-based dyes such as phenyl and substituted-phenyl moieties. Most preferred dyes are rhodamine, fluorescein, dansyl, naphthalene and derivatives thereof. The choice of the dye attached to the chelating moiety will determine the metal ion-binding compound's absorption and fluorescence emission properties as well as its live cell properties, i.e. ability to localize to mitochondria.

Selected sulfonated labels also exhibit advantageous properties, such as solubility, and include sulfonated pyrenes, coumarins, carbocyanines, and xanthenes (as described in U.S. Pat. Nos. 5,132,432; 5,696,157; 5,268,486; 6,130,101). Sulfonated pyrenes and coumarins are typically excited at wavelengths below about 450 nm (U.S. Pat. Nos. 5,132,432 and 5,696,157). Sulfonated Alexa Fluor dyes are particularly preferred.

Fluorescent proteins also find use as reactive labels for conjugation to a carrier molecule or solid support. Examples of fluorescent proteins include green fluorescent protein (GFP) and the phycobiliproteins and the derivatives thereof. The fluorescent proteins, especially phycobiliproteins, are particularly useful for creating tandem dye-reporter molecules. These tandem dyes comprise a fluorescent protein and a fluorophore for the purposes of obtaining a larger Stokes shift, wherein the emission spectra are farther shifted from the wavelength of the fluorescent protein's absorption spectra. This property is particularly advantageous for observing a low quantity of a target analyte in a sample wherein the emitted fluorescent light is maximally optimized; in other words, little to none of the emitted light is reabsorbed by the fluorescent protein. For this to work, the fluorescent protein and fluorophore function as an energy transfer pair wherein the fluorescent protein emits at the wavelength that the acceptor fluorophore absorbs and the fluorophore then emits at a wavelength farther from the fluorescent proteins than could have been obtained with only the fluorescent protein. Alternatively, the fluorophore functions as the energy donor and the fluorescent protein is the energy acceptor. Particularly useful fluorescent proteins are the phycobiliproteins disclosed in U.S. Pat. Nos. 4,520,110; 4,859,582; 5,055,556 and the fluorophore bilin protein combinations disclosed in U.S. Pat. No. 4,542,104. Alternatively, two or more fluorophore dyes can function as an energy transfer pair wherein one fluorophore is a donor dye and the other is the acceptor dye including any dye compounds disclosed in U.S. Pat. Nos. 6,358,684; 5,863,727; 6,372,445; 6,221,606; 6,008,379; 5,945,526; 5,863,727; 5,800,996; 6,335,440; 6,008,373; 6,184,379; 6,140,494 and 5,656,554.

In the context of the present invention, a nanocrystal could be considered either as a reactant A, a carrier species or as a reactant B, a labeling species. Thus, there exist cases, where it is desirable to modify in a controlled manner the surface of a nanocrystal with numerous labels; in this event the nanocrystal is properly conceived as a carrier species, A. For instance, in certain cases it is important to be able to modify and control the number of labels attached to the surface of a core-shell CdSe/ZnS nanocrystal coated on its exterior with reactive groups such as primary carboxylic acid or primary amine functionalities. Examples of this exist in uses of nanocrystals as carriers in biosensor applications, where non-covalent binding of a target species to the surface of the label-modified particle can result in an optically observable signal [Medintz, et al. Proc. Natl. Acad. Sci. USA 2004 101(26): 9612-9617.]

On the other hand, when a nanocrystal is attached to a molecular recognition element such as an antibody or other biological species or receptor, it is more properly considered as B, a labeling species, in analogy with a “standard” fluorescent dye. Numerous examples exist of simple use of nanocrystals as light emitting tags, for example tracers within living cells and within living organisms, such as a Qdot™ nanocrystal product (Invitrogen Corporation) [Michalet, X. et al., Science 2005 307(5709): 538-544; US-2003-0059635; U.S. Pat. Nos. 6,680,211; 6,761,877 and 6,179,912].

Fluorescent nanocrystals can be semiconductor nanocrystals or doped metal oxide nanocrystals. Nanocrystals typically are comprised of a core comprised of at least one of a Group II-VI semiconductor material (of which ZnS, and CdSe are illustrative examples), or a Group III-V semiconductor material (of which GaAs is an illustrative example), a Group IV semiconductor material, or a combination thereof. The core can be passivated with a semiconductor overlayering (“shell”) uniformly deposited thereon. For example, a Group II-VI semiconductor core may be passivated with a Group II-VI semiconductor shell (e.g., a ZnS or CdSe core may be passivated with a shell comprised of YZ wherein Y is Cd or Zn, and Z is S, or Se). The nanocrystals can be operably bound to, and functionalized by the addition of, a plurality of molecules which provide the functionalized fluorescent nanocrystals with reactive functionalities. Nanocrystals can be soluble in an aqueous-based environment. An attractive feature of semiconductor nanocrystals is that the spectral range of emission can be changed by varying the size of the semiconductor core.

An analogous rationale, nanocrystals as labels, can be applied to controlled surface modification of other types of particles, such as small gold and silver particles used in labeling and detection applications. An example is resonance light scattering (RLS) particles, which have demonstrated uses in high sensitivity microarray and bioassay work [Yguerabide, J. and Yguerabide, E E, 2001 J. Cell Biochem Suppl. 37: 71-81; U.S. Pat. Nos. 6,214,560; 6,586,193 and 6,714,299].

### Reactive Groups

The labels of the present invention further comprise a reactive group for the purpose of forming a covalent bond during a conjugation reaction to a carrier molecule or solid support. The labels of the present invention are chemically reactive as are the reactive label competitors as well as the carrier molecule and solid support wherein these substances contain a reactive group or functional group. Reactive or functional groups are typically either a nucleophile, an electrophile or a photoactivatable group wherein an appropriate matching of an electrophile on one compound and a nucleophile on another compound, under appropriate conditions, will form a covalent bond. Photoactivatable groups can form a covalent bond when illuminated with an appropriate wavelength.

These reactive groups are synthesized during the formation of the label, and typically during some stage of synthesis or development of a carrier molecule or solid support. During a labeling reaction a reactive label will form a covalent bond with a carrier molecule or solid support, and in the case of the present invention, with the reactive label competitor when present. In this instance the reactive dye comprises a reactive group (e.g. nucleophile) that will react with a group (e.g. electrophile) on the carrier molecule or solid support. The reactive label competitor does not need to comprise the same reactive group as the carrier molecule or solid support. Preferably, but not necessarily, the reactive group of the reactive label competitor will also be reactive with the reactive label with the same kinetics as its reaction with the carrier molecule or solid support. In one embodiment the reactive group is the same. In another embodiment the reactive group is the same class, but not identical in chemical composition. Selected examples of functional groups and linkages are shown in Table 1, where the reaction of an electrophilic group and a nucleophilic group yields a covalent linkage.

TABLE 1
Examples of some routes to useful covalent linkages

| | | Resulting Covalent |
|---|---|---|
| Electrophilic Group | Nucleophilic Group | Linkage |
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| | | anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |
| Inorganic azide or alkyl azine | phosphine | amide bond |

*Activated esters, as understood in the art, generally have the formula —COΩ, where Ω is a good leaving group (e.g., succinimidyloxy (—OC₄H₄O₂) sulfosuccinimidyloxy (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates

Typically, the conjugation reaction between the reactive group on the label and the carrier molecule or solid support results in one or more atoms of the reactive group being incorporated into a new linkage attaching the label to the carrier molecule or solid support. Typically, the reactive group is separated from the label (or carrier molecule, solid support or reactive label competitor) by a linker.

The resulting bond between, for example, a label and a carrier molecule may be a single bond (where Linker is a single bond) or a series of stable bonds (where the linker contains multiple nonhydrogen atoms). When the linker is a series of stable covalent bonds the linker typically incorporates 1-30 nonhydrogen atoms selected from the group consisting of C, N, O, S and P. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. Typically the linker incorporates less than 15 nonhydrogen atoms and are composed of any combination of ether, thioether, thiourea, amine, ester, carboxamide, sulfonamide, hydrazide bonds and aromatic or heteroaromatic bonds. Typically the linker is a combination of single carbon-carbon bonds and carboxamide, sulfonamide or thioether bonds. The bonds of the linker typically result in the following moieties that can be found in the linker: ether, thioether, carboxamide, thiourea, sulfonamide, urea, urethane, hydrazine, alkyl, aryl, heteroaryl, alkoky, cycloalkyl and amine moieties. Examples of a linker include substituted or unsubstituted polymethylene, arylene, alkylarylene, arylenealkyl, or arylthio.

In one embodiment, the linker contains 1-6 carbon atoms; in another, the linker comprises a thioether linkage. Exemplary linking members include a moiety that includes —C(O)NH—, —C(O)O—, —NH—, —S—, —O—, and the like. In a further embodiment, the linker is or incorporates the formula —O—(CH₂)—. In yet another embodiment, the linker is or incorporates a phenylene or a 2-carboxy-substituted phenylene.

An important feature of the linker is to provide an adequate space between the label and the carrier molecule or solid support so as to prevent steric hindrance. This is particularly important when relatively small protein molecules, such as Fab′ fragments, are being labeled with more than one dye. Typically the DOL is in the 2-5 range.

In an exemplary embodiment, the labels comprise a reactive group that comprises an acrylamide, an activated ester of a carboxylic acid, a carboxylic ester, an acyl azide, an acyl nitrile, an aldehyde, an alkyl halide, an anhydride, an aniline, an amine, an aryl halide, an azide, an aziridine, a boronate, a diazoalkane, a haloacetamide, a haloalkyl, a halotriazine, a hydrazine, an imido ester, an isocyanate, an isothiocyanate, a maleimide, a phosphoramidite, a photoactivatable group, a reactive platinum complex, a silyl halide, a sulfonyl halide, and a thiol. In a particular embodiment the reactive group comprises carboxylic acid, succinimidyl ester of a carboxylic acid, hydrazide, amine and a maleimide.

In one aspect, the reactive group selectively reacts with an amine group. This amine-reactive group, includes but is not limited to, succinimidyl ester (SE), sulfonyl halide, tetrafluorophenyl ester or iosothiocyanates. Thus, in one aspect, the labels form a covalent bond with an amine containing molecule in a sample. In another aspect, the label comprises at least one reactive group that selectively reacts with a thiol group. This thiol-reactive group includes, but is not limited to, a maleimide, haloalkyl or haloacetamide (including any reactive groups disclosed in U.S. Pat. Nos. 5,362,628; 5,352,803 and 5,573,904).

The choice of the reactive group used to covalently conjugate the label to the carrier molecule or solid support typically depends on the reactive or functional group on this molecule or support and the type or length of covalent linkage desired. The types of functional groups typically present on the organic or inorganic substances (biomolecule or non-biomolecule) include, but are not limited to, amines, amides, thiols, alcohols, phenols, aldehydes, ketones, phosphates, imidazoles, hydrazines, hydroxylamines, disubstituted amines, halides, epoxides, silyl halides, carboxylate esters, sulfonate esters, purines, pyrimidines, carboxylic acids, olefinic bonds, or a combination of these groups. A single type of reactive site may be available on the substance (typical for polysaccharides or silica), or a variety of sites may occur (e.g., amines, thiols, alcohols, phenols), as is typical for proteins.

Typically, the reactive group will react with an amine, a thiol, an alcohol, an aldehyde, a ketone, or with silica silanol groups. Preferably, reactive groups react with an amine or a thiol functional group, or with silica silanol groups. In one embodiment, the reactive group is an acrylamide, an activated ester of a carboxylic acid, an acyl azide, an acyl nitrile, an aldehyde, an alkyl halide, a silyl halide, an anhydride, an aniline, an aryl halide, an azide, an aziridine, a boronate, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as described in U.S. Pat. No. 5,714,327.

Where the reactive group is an activated ester of a carboxylic acid, such as a succinimidyl ester of a carboxylic acid, a sulfonyl halide, a tetrafluorophenyl ester or an isothiocyanates, the resulting compound is particularly useful for preparing conjugates of carrier molecules such as proteins, nucleotides, oligonucleotides, or haptens. Where the reactive group is a maleimide, haloalkyl or haloacetamide (including any reactive groups disclosed in U.S. Pat. Nos. 5,362,628; 5,352,803 and 5,573,904 (supra)) the resulting compound is particularly useful for conjugation to thiol-containing substances. Where the reactive group is a hydrazide, the resulting compound is particularly useful for conjugation to periodate-oxidized carbohydrates and glycoproteins, and in addition is an aldehyde-fixable polar tracer for cell microinjection. Where the reactive group is a silyl halide, the resulting compound is particularly useful for conjugation to silica surfaces, particularly where the silica surface is incorporated into a fiber optic probe subsequently used for remote analyte detection or quantitation.

In a one aspect, the reactive group is a photoactivatable group such that the group is only converted to a reactive species after illumination with an appropriate wavelength. An appropriate wavelength is generally a UV wavelength that is less than 400 nm. This method provides for specific attachment to only the target molecules, either in solution or immobilized on a solid or semi-solid matrix. Photoactivatable reactive groups include, without limitation, benzophenones, aryl azides and diazirines.

Preferably, the reactive group is a succinimidyl ester of a carboxylic acid, a haloacetamide, haloalkyl, a hydrazine, an isothiocyanate, a maleimide group, an aliphatic amine, a silyl halide, a cadaverine or a psoralen. More preferably, the reactive group is a succinimidyl ester of a carboxylic acid, a maleimide, an iodoacetamide, or a silyl halide. In a particular embodiment the reactive group is a succinimidyl ester of a carboxylic acid, a sulfonyl halide, a tetrafluorophenyl ester, an iosothiocyanates or a maleimide.

### Reactive Label Competitor (Reactant C)

The reactive label competitor is any compound that reacts in the same manner, under the same reaction conditions, as reactant A does with reactant B, only reactant C forms a product with B (BC) removing a portion of B from the reaction so that there is less to react with reactant A. Typically the reactive label competitor comprises the same reactive or functional group as reactant A.

In one embodiment, the carrier molecule or solid support comprises an amine group and the reactive label comprises an amine-reactive group. In this instance a preferred reactive label competitor would also comprise an amine group.

In one embodiment the reactive label competitor comprises α- and β-amino acids, amino alcohols, ε-amino acids, primary amines and reactive secondary amine-containing compounds. In a further aspect the reactive label competitor comprises D-, L-, or D,L-lysine, ethanoloamine and; 5-amino caproic acid or, ammonia (NH₃). In a particular aspect the reactive label competitor is L-lysine HCl.

An example of the use of lysine would be addition of L-lysine HCl to a reaction solution in which a protein (reactant A) is reacting with a reactive label that comprises an activated succinimidyl ester (SE). It is well known that the free ε amino group of lysine reacts selectively with SE esters to form stable amide adducts, whether the free ε amino group belongs to a lysine accessible on the surface of the protein polypeptide or whether the ε amino group resides on added soluble free lysine. Provided the proper concentration of lysine is included in the solution at the beginning of the reaction period, the net result of the presence of the lysine modulator is a predictable decrease in the overall DOL in the final product, compared with the DOL that would occur in the absence of the modulator species C. In this example, the reaction rate of the free lysine modulator with the active SE ester is affected by largely the same factors (pH, temperature, concentration, etc) as affect the reaction rate of the lysines residing in the protein polypeptide. An important feature of the present invention is that the concentration of the reactive label competitor (reactant C) during the reaction can be varied to alter its reaction rate with the activated reactive label (reactant B), to provide conditions where up to a several-fold molar excess of reactant C over reactant B can be utilized. Relatively slow, controlled reaction of the reactive label competitor (reactant C) with the reactive label (reactant B) to give product BC results in sufficient remaining activated reactive label B in solution to, over time, result in derivatization of reactant A (carrier molecule or solid support), but resulting in a smaller final DOL, and in a relatively predictable manner.

In another embodiment, carrier A is a macromolecule containing a covalently bound azide group or groups, reactive label B is a phosphine-based chemical, and the reactive label competitor is a chemical species that can react with the phosphine, such as inorganic azide or an alkyl azide (Staudinger-type chemistry). The azide groups located in the carrier can be chemically or enzymatically incorporated in vitro, or incorporated by the cellular biochemical machinery in vivo (US publication No. 2005/0148032). The phosphine-based reactive species may be, for instance, a dye or metal chelate derivative which is capable of reacting selectively with azides present either on carrier A or with the reactive label competitor to effect control of DOL.

In another embodiment, the carrier molecule or solid support comprises a thiol group and the reactive label comprises a thiol-reactive group. In this instance a preferred reactive label competitor would also comprise a thiol group.

In one embodiment the reactive label competitor comprises α- and β-mercapto acids, mercapto alcohols, ε-mercapto acids, primary mercaptans and reactive secondary mercaptan compounds. In a further aspect the reactive label competitor comprises D-, L-, or D,L-cysteine, mercaptoethanol, 5-mercapto caproic acid or, H₂S.

In an example, a reduced reactive sulfhydryl (—S—) group associated with a protein, for example resulting from reduction of disulfide-linked (S—S) sulfhydryls in an antibody (reactant A), reacts with a sulfhydryl-selective compound such as a maleimide derivative (reactant B). In this case the modulator (C—S—) could be a sulfhydryl compound added to react with portion of reactant B, thus modulating the reaction of A with B (Eq. 1):

A-S⁻+B->A-S—B(labeled antibody)+C—S⁻->C—S—B(inactivated B) (Eq. 1)

In a preferred embodiment, the reactive label competitor is a single small molecule species; but another embodiment of the reactive label competitor may comprise a larger molecular or macromolecular assembly that bears groups able to react with the reactive label. In an example, the reactive label competitor may be a protein bearing multiple reactive sites, such lysine ε-amino groups, or the reactive label competitor may be another type of polymer, such as a modified ribonucleic or deoxyribonucleic acid polymer or other type of natural or synthetic polymer bearing suitable reactive groups.

Protein derivatization efficiency is typically dependent on the concentration of protein in the derivatization reaction. Typically, optimization of dye concentration is required when trying to control the degree of labeling of the target protein. One embodiment of the present invention provides a method and composition for achieving the same degree of protein derivatization independent of the concentration of the protein. The method is based on the addition of reactive label competitor (or an attenuator) which controls both the rate and extent of reaction of the dye.

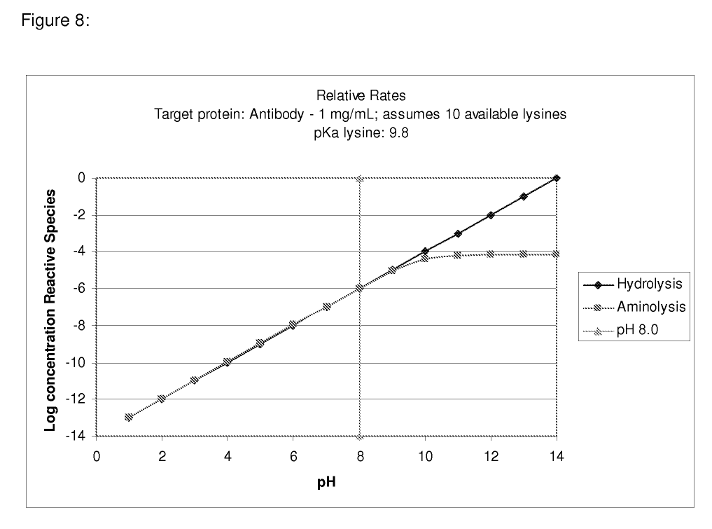

In a buffered solution, the hydrolysis rate of a reactive label is essentially constant in that the [OH⁻] does not appreciably change as the reaction proceeds, so long as the pH of the reaction remains constant. When considering derivatization of protein amines, protein derivatization efficiency is generally dictated by the relative rate of aminolysis (reaction with the deprotonated amine) and hydrolysis (reaction with the hydroxide ion). Efficient protein derivatization occurs when the relative rate of aminolysis is significantly greater than hydrolysis. The pH dependent-concentrations of deprotonated amine and hydroxide ion and fraction of the total reactive species are shown in FIG. 8. The concentration of deprotonated amine is calculated using the Henderson-Hasselbach equation. As can be seen in the figure, the relative concentrations of the reactive amines and hydroxide ion concentrations track each other until close to the pKₐ of lysine. At a pH close to lysine pKₐ, the concentration of reactive lysines (deprotonated amines) begin to reach saturation at the concentration of amines present in the reaction. A similar figure can be drawn for any reactive label competitor where the point of saturation will be dependent on the pKₐ of the species.

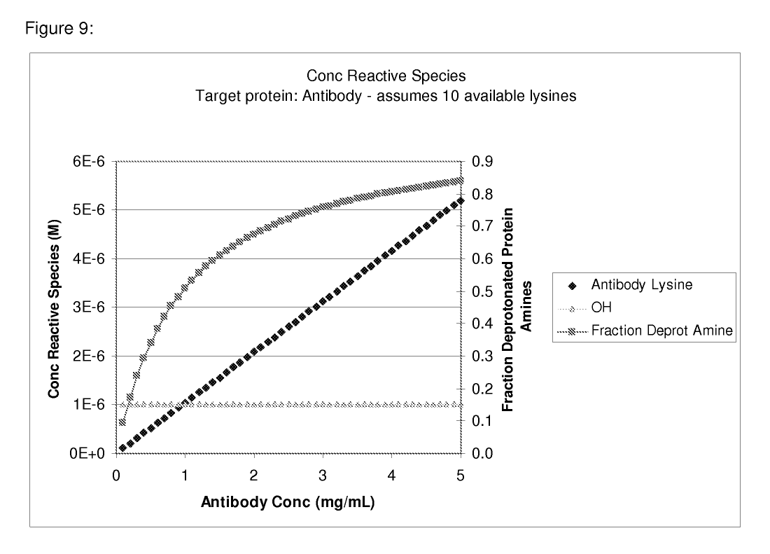

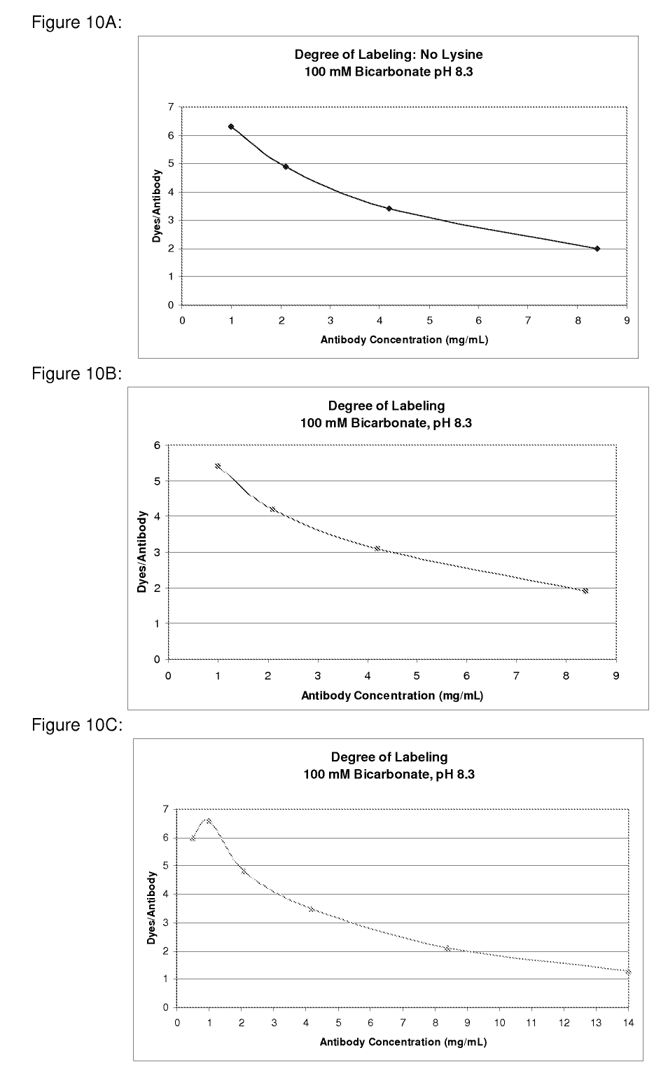

The relative concentrations of deprotonated protein amines and hydroxide ions are a function of protein concentration in a reaction occurring at pH 8.0 (FIG. 9). The figure shows that the proportion of reactive species (Amines and hydroxides) contributed by the lysines is not a linear function of the added protein. Since the aminolysis and hydrolysis rates are proportional to the deprotonated protein amine and hydroxide ion concentrations, respectively, at low protein concentrations, hydrolysis predominates. At high protein concentrations, aminolysis predominates. As a result, protein derivatization efficiency varies with the concentration of target protein. The protein concentration-dependence is observed experimentally (FIG. 10).

Despite the dependence of protein derivatization efficiency on protein concentration, the efficiency of protein derivatization can be predicted knowing the reaction composition. The fraction of reactive species contributed by the target protein is expressed as in Eq. 2:

$$Fraction ProteinAmines = \frac{[ Deprotonated ProteinAmines ]}{[ DeprotonatedPr oteinAmines ] + [ {OH}^{-} ]} \qquad ( Eq . 2 )$$

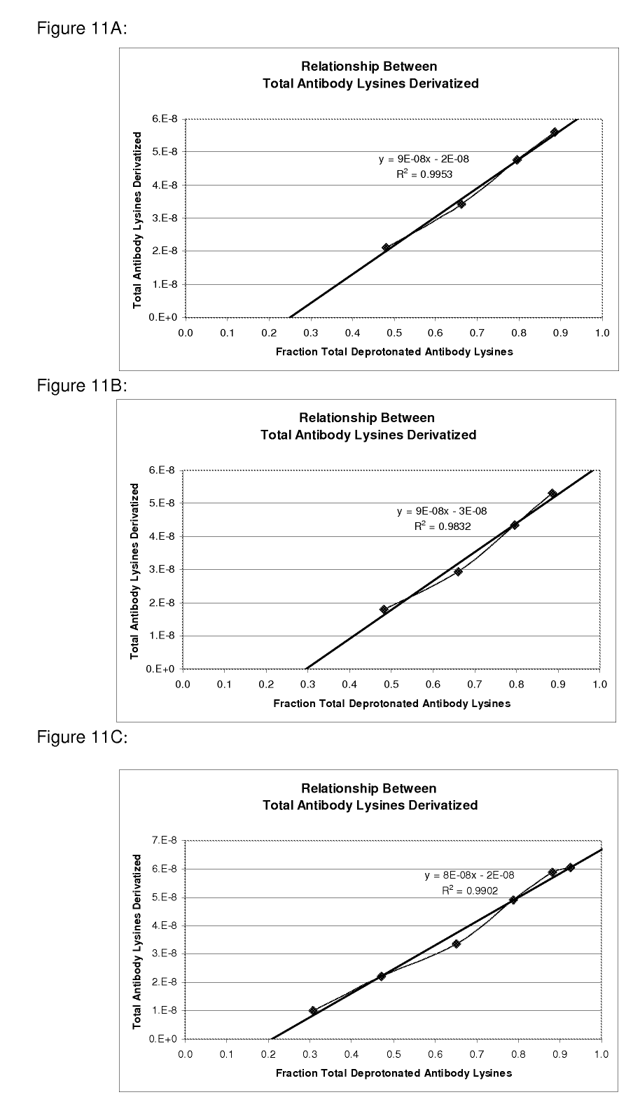

The results of several experiments demonstrate that the protein derivatization efficiency can be predicted from the fraction of reactive species contributed by the target protein (FIG. 11). This result is very reproducible as seen in three separate experiments (FIG. 11: A, B, and C).

The above analysis assumes that the only reactive species in proteins are lysines. However, it is known that histidine imidazoles and tyrosines react with activated acyl groups to generate unstable intermediates which undergo subsequent hydrolysis. Therefore, there is an antibody concentration-dependent component to the overall hydrolysis rate. The contribution of histidine to the overall reaction can be significant since the histidine imidazole has a pKₐ of 6.95 while antibody lysines have a pKa of 9.8. At pH 8.0, 92% and 1.6% of histidines and lysines are unprotonated, respectively. The relative ratio of deprotonated histidines relative to deprotonated lysines has been demonstrated. The relative contribution of histidines to apparent hydrolysis and protein lysines to aminolysis is dependent on the relative reaction rates of the two species with the activated dye. It has been shown that lysines are more reactive with active esters compared to imidazole. In addition, aliphatic alcohols such as serine and threonine can slowly react with active esters.

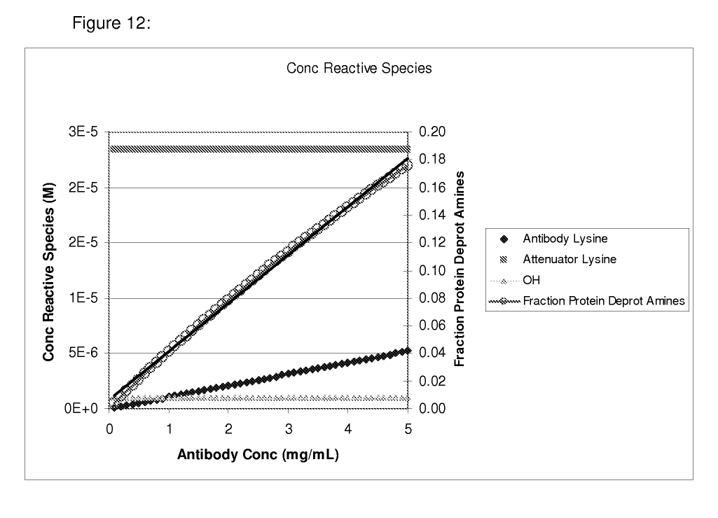

As shown in FIG. 9, the rate of reaction of deprotonated amines on the protein with reactive label is a function of protein concentration. It can be seen that the proportion of reactive species contributed by the amines is not a linear function of the added protein concentration because addition of increasing concentrations of protein results in a change in the total reactive species present. However, upon addition of an excess amount of reactive label competitor, such as lysine, the proportion of reactive species contributed by the protein amines increases linearly as a function of protein concentration (FIG. 12). This is because increasing the concentration of protein does not significantly contribute to the total number of reactive species present.

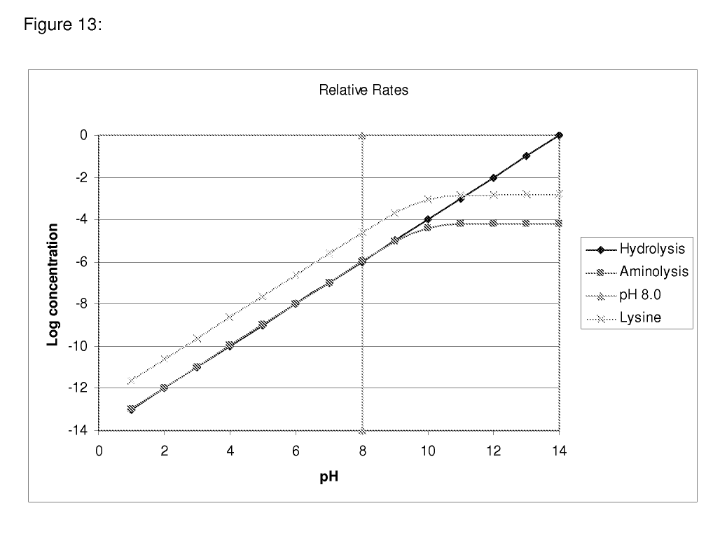

Depending on the pKₐ of the reactive label competitor species, the contribution of the protein to the overall reactive species will track over a wide range of pHs (FIG. 13). Alternative reactive label competitor species are also possible, including, but not limited to, primary amines, secondary amines, tertiary amines, aliphatic alcohols, aromatic alcohols. The reactive label competitor species concentration must be adjusted such that the reactive species is in significant excess over the concentration of the added protein reactive groups.

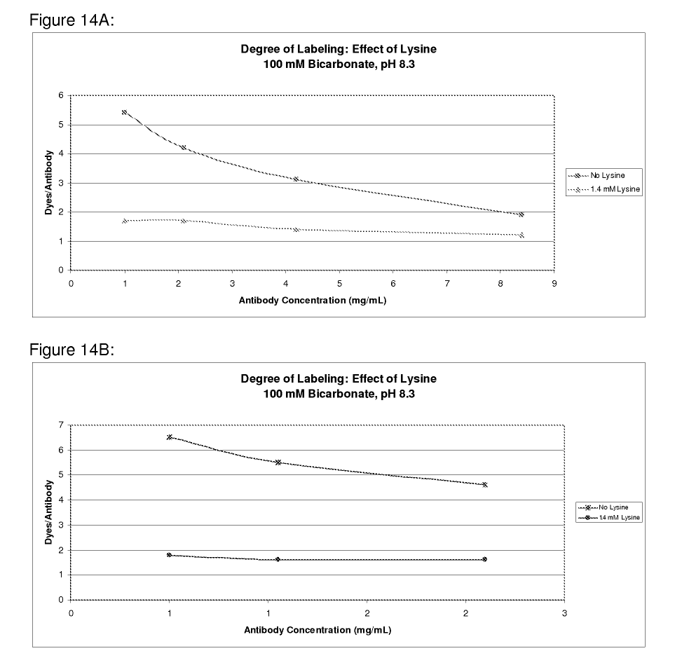

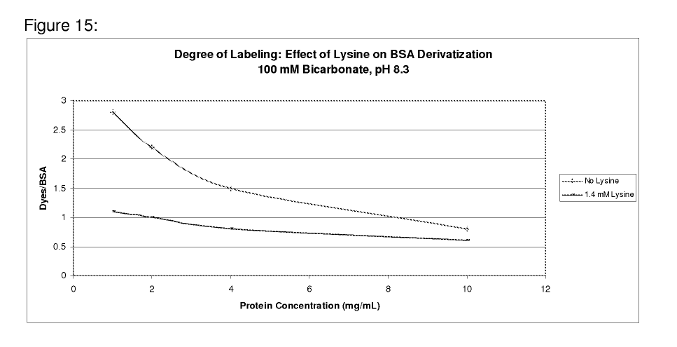

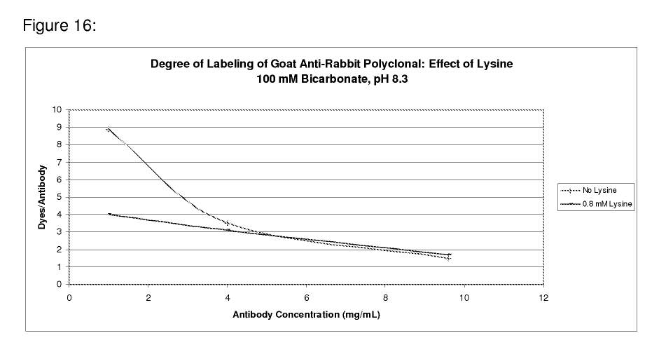

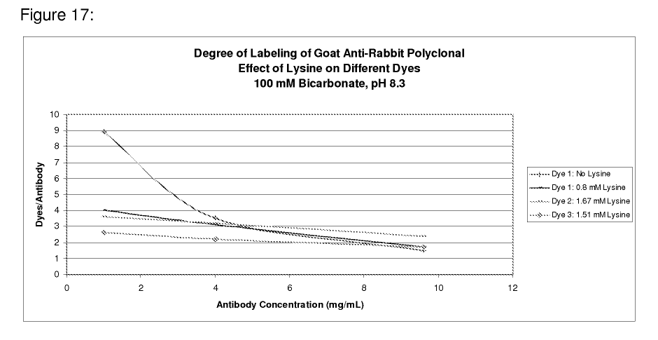

The above models are supported by FIG. 14. The effect of reactive label competitor addition is not limited to monoclonal IgG antibodies. A similar effect can be seen derivatizing BSA (FIG. 15) and a goat polyclonal IgG antibody (FIG. 16). Similar data was found using active esters of three different dyes (FIG. 17).

The addition of reactive label competitor to generate nearly protein concentration-independent derivatization efficiency is not limited to NHS active esters or the reaction of amines. Similar approaches can be taken using active esters, aldehydes, imidates, imidoesters, isothiocyanates, aryl halides, acylazides, alkyl halides, etc. The method is best applied when trying to control the effect of competing reactions on protein derivatization efficiency.

### Methods of Use

The present invention provides methods for modulating the amount of reactive label and thereby controlling, in predictable manner, the DOL of a carrier molecule or solid support by a reactive label when a reactive label competitor is present during the conjugation reaction.

One embodiment of the present invention provides a method of modulating the amount of reactive label present in a solution, said method comprising:
a) contacting a solution comprising a carrier molecule or solid support with a reactive label to form a labeled carrier molecule or labeled solid support; and
b) contacting the solution with a reactive label competitor to form a labeled competitor;
wherein the amount of reactive label in the solution is attenuated or eliminated after contacting the reactive label with the reactive label competitor.

In another embodiment the method of modulating the amount of reactive label present in a solution, controls the degree of labeling of the carrier molecule or solid support.

In another embodiment the amount of labeled carrier molecule or labeled solid support is essentially unaffected by the concentration of carrier molecule or solid support in solution. In another embodiment thereof, the pH is between 3 and 10. More particularly, the pH is between 7 and 9. More particular still, the pH is between 8 and 9.

In another embodiment, the reactive label is a reactive dye. In another embodiment, the reactive label is a reactive biotin. In another embodiment, the reactive label is a reactive ligand.

In another embodiment, a reactive group on the reactive label is an active ester, an aldehyde, an alkyl halide, an imidate, an imidoester, an isothiocyanate, an aryl halide or an acylazide.

In another embodiment, the reactive label competitor contains a primary amine, a secondary amine, a tertiary amine, an aliphatic alcohol or an aryl alcohol. In another embodiment, the reactive label competitor is lysine, imidazole, histidine, tyrosine, serine, threonine, enthanolamine, ethylamine, or propylamine.

The reactive label and the reactive label competitor are optionally added to the solution simultaneously, or separately. In one embodiment the reactive label competitor is added after contacting a solution comprising a carrier molecule or solid support with the reactive label competitor. In another embodiment the reactive label competitor is added to the solution comprising a carrier molecule or solid support before contacting the solution with the reactive label competitor. In another embodiment, the method of modulating the amount of reactive label present in solution comprises a one-pot solution, wherein the reactive label, reactive label competitor and carrier molecule or solid support are added to the solution at approximately the same time, wherein the labeled competitor and labeled carrier molecule or labeled solid support are both formed in the same solution.

Another more particular embodiment further comprises a step of separating labeled competitor from the labeled carrier molecule or labeled solid support. More particular still, the step of separating comprises column chromatography. In another embodiment, the reactive label competitor is biotin, hexahistidine, dignoxigenin, a positively charged group, a negatively charged group, or a large molecular weight species.

The separating step improves removal of reaction byproducts from the target derivatization reaction. In a typical dye labeling reaction, the products include: underivatized protein, dye-target conjugate, hydrolyzed dye, and free leaving group. Dye-target and hydrolyzed dye are the only labeled species present in the product. If not properly removed, the presence of hydrolyzed dye can increase background complicating the use of the dye-target conjugate.

The presence of the reactive label competitor significantly reduces the amount of free dye resulting from hydrolysis. The composition of the reaction following derivatization in the presence of reactive label competitor is:
1. Underivatized protein
2. Dye-target conjugate
3. Dye-reactive label competitor conjugate (labeled competitor)
4. Free reactive label competitor
5. Free leaving group
6. Hydrolyzed label

In this case, dye-target and labeled competitor conjugates are the predominant species present in the product mixture.

The role of purification following target labeling is predominantly to remove hydrolyzed dye as it interferes with subsequent analysis using the labeled target. Size exclusion chromatography is typically used to separate the low molecular weight hydrolyzed dye from dye conjugated to protein. Size exclusion chromatography is generally labor intensive and slow. In addition, size exclusion chromatography will be inefficient if the molecular weight of the labeled target is similar to that of the hydrolyzed dye. This is true when derivatizing low molecular weight compounds.

Addition of the reactive label competitor essentially eliminates most of the products resulting from hydrolysis. The structure of the reactive label competitor can be designed to aid in subsequent purification.

As described above, the reactive group can be any group that acts as a competitor for the derivatization reaction. The purification group can be any group that will aid in subsequent purification. For example, if the purification group is biotin, efficient removal of the labeled competitor can be achieved by passing the conjugated reaction over immobilized streptavidin or biotin. Alternative purification groups can be used. The major feature is that the purification group distinguishes the dye-reactive label competitor conjugate from that of the dye-target conjugate. For example, if the dye-target is positively charged, the purification group could be designed to introduce negative charges into the dye-reactive label competitor product. Such an approach would allow rapid removal of the dye-reactive label competitor product using electrophoretic or ion exchange means. If the dye-target product was low in molecular weight, the reactive label competitor purification group could introduce a large molecular weight species (such as PEG) to clearly distinguish the dye-reactive label competitor product from the dye-target product. Rapid separation methods, such as smaller sizing columns or electrophoresis methods could then be used to easily remove the dye-reactive label competitor product.

Another embodiment provides a method for controlling the degree of labeling (DOL) of a carrier molecule or solid support, wherein the method comprises:
a) contacting the carrier molecule or solid support with a reactive label to form a labeling solution;
b) contacting the labeling solution with a reactive label competitor to form a controlled labeling solution; and
c) incubating the controlled labeling solution for an appropriate amount of time whereby the degree of labeling of the carrier molecule or solid support is controlled.

Altering the DOL to what may be an acceptable level may be achieved by altering the reaction conditions under which the carrier molecule is labeled by the reactive dye. An effective way to achieve this is to include a competitor in the reaction solution that competes, for example, with the carrier-bound reactive amines for reaction with the reactive label. The competitor can be any reactive group that can be added to the reaction solution in sufficient amounts in a controlled manner to allow partial quenching of the reaction of the reactive label with the carrier-bound amines, most probably the ε amino group of lysines on a protein. The competitor may or may not react in an instantaneous manner with the reactive label, depending upon the chemical reaction kinetics of the system. The competitor may in fact be added to a total initial concentration greater than either the carrier molecule or the reactive label, as long as reaction kinetics obtain in which the antibody-bound amines continue to react at a rate that results in net derivatization of the protein. Conveniently, the competitor may be added in an appropriate concentration at the beginning of the labeling reaction, or it is possible to add it to the reaction solution at some time after the carrier-reactive label reaction has begun. Careful titration of the reaction solution with relatively small volumes of concentrated competitor represents a robust and reproducible means of controlling the DOL, while keeping the reaction volume nearly constant. This in turn allows standardized purification methods to be used across a range of reactions and final DOL values.

Another aspect of the present invention provides method for monitoring the degree of labeling (DOL) of a carrier molecule or solid support, said method comprising:
a) contacting a solution comprising a carrier molecule or solid support with a reactive label to form a labeled carrier molecule or labeled solid support; and
b) contacting the solution with a reactive label competitor to form a labeled competitor, wherein the reactive label competitor quenches or is capable of FRET interaction with the reactive label;
wherein the degree of labeling (DOL) is monitored by the amount of quenching or FRET that occurs between the label and the reactive label competitor.

Accordingly, another aspect of this invention is to provide a way for monitoring and quantifying the labeling reaction. Because addition of the reactive label competitor results in predominantly two products, the reaction can be monitored by adding a signaling group to the reactive label competitor constructs. Additionally, the solution can contain a pH buffer.

The signaling group can either be a quencher that quenches the fluorescence of the dye or a fluorophore capable of undergoing FRET with the dye used in the conjugation reaction. Thus, the products of the reaction will be:
1. Underivatized protein
2. Dye-target conjugate
3. Dye-reactive label competitor-signaling group conjugate
4. Free reactive label competitor-signaling group
5. Free leaving group

If the signaling group consists of a quencher, only the dye-target conjugate will fluoresce. As a result, quantification of the degree of protein derivatized can be assessed by measuring the total fluorescence of the reaction prior to purification.

If the signaling group consists of a fluorophore capable of FRET interaction with the conjugating fluorophore, quantification of the degree of protein derivatization can be assessed by measuring at the amount of fluorescence not undergoing FRET.

In both cases, monitoring the decrease in fluorescence (in the case the signaling group is a quencher) or an increase in FRET (in the case the signaling group is a fluorophore) will allow monitoring both the rate and the completion of the derivatization reaction.

It is well known in the art methods for forming conjugate between reactive labels and either carrier molecules or solid supports. The present methods do not alter those methods but instead include the addition of a supplement reactant without altering those initial reactants. Thus, any method known to one of skill in the art can be used to form a labeled conjugate wherein the addition of a reactive label competitor allows the end user to produce product with a desired DOL. See, Examples 1-4.

Provided in another embodiment are conjugates formed by using the present method to control the DOL. Thus, is provided labeled carrier molecule or solid support conjugate comprising a controlled DOL made by a process comprising:
a) contacting the carrier molecule or solid support with a reactive label to form a labeling solution;
b) contacting the labeling solution with a reactive label competitor to form a controlled labeling solution; and
c) incubating the controlled labeling solution for an appropriate amount of time whereby the carrier molecule or solid support is made with a controlled DOL.

Conjugates of components (carrier molecules or solid supports), e.g., drugs, peptides, toxins, nucleotides, phospholipids and other organic molecules are prepared by organic synthesis methods using the reactive labels of the invention, are generally prepared by means well recognized in the art (Haugland, MOLECULAR PROBES HANDBOOK, supra, (2002)). Preferably, conjugation to form a covalent bond consists of simply mixing the reactive labels of the present invention in a suitable solvent in which both the reactive label and the substance to be conjugated are soluble. The reaction preferably proceeds spontaneously without added reagents at room temperature or below. Conjugation reactions performed at room temperature typically proceed to completion within about 2 hours, more typically within about 1 hour or 60 minutes. Those conjugation reactions performed on ice typically proceed to completion within about 24 hours, more typically within about 20 hours.

For those reactive labels that are photoactivated, conjugation is facilitated by illumination of the reaction mixture to activate the reactive label. Chemical modification of water-insoluble substances, so that a desired label-conjugate may be prepared, is preferably performed in an aprotic solvent such as dimethylformamide, dimethylsulfoxide, acetone, ethyl acetate, toluene, or chloroform. Similar modification of water-soluble materials is readily accomplished through the use of the instant reactive compounds to make them more readily soluble in organic solvents.

Preparation of Peptide or Protein Conjugates Typically Comprises First Dissolving the Protein to be conjugated in aqueous buffer at about. 1-10 mg/mL at room temperature or below. Bicarbonate buffers (pH about 8.3) are especially suitable for reaction with succinimidyl esters, phosphate buffers (pH about 7.2-8) for reaction with thiol-reactive functional groups and carbonate or borate buffers (pH about 9) for reaction with isothiocyanates and dichlorotriazines. The appropriate reactive label is then dissolved in a nonhydroxylic solvent (usually DMSO or DMF) in an amount sufficient to give a suitable degree of conjugation when added to a solution of the protein to be conjugated, typically within a range such as 4-8 that can then be modified with the reactive label competitor such that a specified number of labels, such as 4, are conjugated to the carrier molecule or solid support. The appropriate amount of label for any protein or other component is conveniently predetermined by experimentation in which variable amounts of the label are added to the protein, the conjugate is chromatographically purified to separate unconjugated compound and the label-protein conjugate is tested in its desired application.

Following addition of the reactive label to the carrier molecule or solid support solution, the mixture is incubated for a suitable period (typically about 1 hour at room temperature to several hours on ice). In the present invention the reactive label competitor is added with the reactive label and carrier molecule or solid support to the reaction solution. After the labeling reaction has proceeded to the desired level of completion the excess label and label-reactive label competitor product are removed by spin columns, gel filtration, dialysis, HPLC, adsorption on an ion exchange or hydrophobic polymer or other suitable means. The label-conjugate is used in solution or lyophilized. In this way, suitable conjugates can be prepared from antibodies, antibody fragments, avidins, lectins, enzymes, proteins A and G, cellular proteins, albumins, histones, growth factors, hormones, and other proteins.

Conjugates of polymers, including biopolymers and other higher molecular weight polymers are typically prepared by means well recognized in the art (for example, Brinkley et al., Bioconjugate Chem., 3: 2 (1992)). In these embodiments, a single type of reactive site may be available, as is typical for polysaccharides or multiple types of reactive sites (e.g. amines, thiols, alcohols, phenols) may be available, as is typical for proteins. Selectivity of labeling is best obtained by selection of an appropriate reactive label. For example, modification of thiols with a thiol-selective reagent such as a haloacetamide or maleimide, or modification of amines with an amine-reactive reagent such as an activated ester, acyl azide, isothiocyanate or 3,5-dichloro-2,4,6-triazine. Partial selectivity can also be obtained by careful control of the reaction conditions.

When modifying polymers with the reactive label, an excess of compound is typically used, relative to the expected degree of label substitution. Any residual, unreacted label, a compound hydrolysis product or a label-reactive label competitor product is typically removed by dialysis, chromatography or precipitation. Presence of residual, unconjugated label or label-reactive label competitor product can be detected by thin layer chromatography using a solvent that elutes the label or label-reactive label competitor product away from its desired conjugate. In all cases it is usually preferred that the reagents be kept as concentrated as practical so as to obtain adequate rates of conjugation.

With the addition of a reactive label competitor to a conjugation reaction, the DOL can be controlled in such a manner that predictable numbers of labels are conjugated to a carrier molecule or solid support. In one embodiment the DOL of a carrier molecule is 6, in a further embodiment the DOL is 5, in a further embodiment the DOL is 4, in a yet a further embodiment the DOL is 3. In certain instances it is important to have a DOL of 2 or even 1 label per carrier molecule.

The change in concentration of the reactive label competitor is the variable, such that when altered, it modulates the number of labels conjugated to a carrier molecule. Thus, as exemplified in Example 3, 1 mg/ml of protein results in a DOL of between 5 and 6 when no competitor (lysine) is added. However, when lysine is added at a concentration of 0.3 mM the DOL is between 3 and 4 labels per molecule. The DOL is further reduced when lysine at a concentration of 1 mM is added resulting in a DOL of about 2 labels per molecule. In this manner, the present invention provides a predictable way in which an end-user can obtain a desired DOL.

In certain aspects altering the DOL is important for a particular aspect. In one embodiment altering the DOL is important for in vivo imaging to reduce the quenching of too many dyes per conjugate and to obtain the brightest observable signal possible.

In another embodiment, reliably altering the DOL is important for localization of labeled carrier molecules in vivo. For example, injected labeled antibodies may predominantly localize on tumor cells but be distributed heterogeneously, and not solely related to expression of cognate antigen and, in some cases, may accumulate in necrotic more than viable areas of a tumor. Chemical and physical differences in antibodies having different DOL values can be important determinants in the occurrence and degree of this heterogeneity [Boxer, G M et al. Br. J. Cancer 1992 65(6): 825-831.]

In another embodiment reliably altering the DOL is important because overlabeling of proteins generally results in altered specificity, aggregation, and/or precipitation of the protein. Fluorescent labeling of antibodies with high fluorophore to antibody ratios (DOL≧˜6) usually results in increased non-specific binding (increased background) and decreased quantum yield due to fluorophore self-quenching.

The resulting label-conjugates of the present invention can be used in all the methods known now, and in the future, to one of skill in the art for using labeled carrier molecules or solid supports; e.g. use of antibody conjugates in microscopy and immunofluorescent assays; and nucleotide or oligonucleotide conjugates for nucleic acid hybridization assays and nucleic acid sequencing (e.g., U.S. Pat. Nos. 5,332,666; 5,171,534; 4,997,928; and WO Appl. 94/05688). Typically labeled conjugates are used to detect, monitor, quantitate, isolate and/or bind a target analyte. Labeled conjugates of multiple independent dyes of the invention possess utility for multi-color applications.

In one embodiment, the labeled conjugate forms a covalent or non-covalent association or complex with an element in a sample, or is simply present within the bounds of the sample or portion of the sample. The sample is then illuminated at a wavelength selected to elicit the optical response. Typically, staining the sample is used to determine a specified characteristic of the sample by further comparing the optical response with a standard or expected response.

A detectable optical response means a change in, or occurrence of, an optical signal that is detectable either by observation or instrumentally. Typically the detectable response is a change in fluorescence, such as a change in the intensity, excitation or emission wavelength distribution of fluorescence, fluorescence lifetime, fluorescence polarization, or a combination thereof. The degree and/or location of staining, compared with a standard or expected response, indicates whether and to what degree the sample possesses a given characteristic.

The optical response is optionally detected by visual inspection, or by use of any of the following devices: CCD cameras, video cameras, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

For biological applications, the labeled conjugates are typically used in an aqueous, mostly aqueous or aqueous-miscible solution prepared according to methods generally known in the art. The exact concentration of dye compound is dependent upon the experimental conditions and the desired results, but typically ranges from about one nanomolar to one millimolar or more. The optimal concentration is determined by systematic variation until satisfactory results with minimal background fluorescence is accomplished.

The labeled conjugates are most advantageously used to stain samples with biological components. The sample may comprise heterogeneous mixtures of components (including intact cells, cell extracts, bacteria, viruses, organelles, and mixtures thereof including small animals), or a single component or homogeneous group of components (e.g. natural or synthetic amino acid, nucleic acid or carbohydrate polymers, or lipid membrane complexes). These labeled conjugates are generally non-toxic to living cells and other biological components, within the concentrations of use.

### C. Kits of the Invention

Due to the advantageous properties and the simplicity of use of the instant reactive label competitors, they are particularly useful in the formulation of a kit for the labeling of a carrier molecule or solid support, comprising one or more reactive labels, reactive label competitor and optionally the carrier molecule or solid support in any of the embodiments described above (optionally in a stock solution), instructions for the use of the competitor, and optionally comprising additional components. In another embodiment the kit comprises a carrier molecule or solid support labeled with a reactive label using the present method of the reactive label competitor and instructions for using the conjugate.

A kit of the present invention for controlling the DOL of a conjugate comprises a present competitor and instructions for use thereof. In a further aspect the kit comprises a reactive label and a carrier molecule or solid support. The kit may further comprise one or more components selected from the group consisting of a purification resin, spin column, collection tubes, a fluorescent standard, an aqueous buffer solution and an organic solvent. The additional kit components are present as pure compositions, or as aqueous solutions that incorporate one or more additional kit components. Any or all of the kit components optionally further comprise buffers.

The examples below are given so as to illustrate the practice of this invention. They are not intended to limit or define the entire scope of this invention.

### EXAMPLES

### Example 1

Use of lysine, a primary amine containing compound, as a reactive label competitor to control the DOL of an Alexa Fluor® 647 dye conjugated to a Goat anti-mouse IgG.

Lysine (L-lysine HCl: SIGMA L5626-500g lot 114k0171) was made as 1 M stock pH adjusted to 8.0 with NaOH, and serial dilutions were made to obtain 0.1, 0.01, 0.0 M stocks, which are sterile filtered and stored at 4° C. The Goat anti-mouse IgG (Fortron Bisocience Inc. Morrisville, N.C.) was diluted with phosphate buffered saline (PBS) to obtain 1 mg/ml stock, stored at 4° C. The labeling was performed according to manufactures instructions (Invitrogen Corp. A20186), with the addition of lysine from stock solutions. For example, in a 1.5 ml tube was combined 100 μl of goat anti-mouse IgG (1 mg/ml), 10 μl 1 M sodium bicarbonate buffer, and 1 to 10 μl of lysine stocks to obtain concentration ranging from 0 to 10 mM lysine. 100 μl of Alexa Fluor 647 dye was added to the 1.5 mL tubes and incubated, in the dark, for 40 minutes.

The labeled goat anti-mouse IgG was purified using a spin column according to manufacturer's instructions (Invitrogen Corp. A20186).

The degree of substitution was determined based on OD readings at A₂₈₀ and A₆₅₀ using a Nanoprop® ND 1000 spectrophotometer. The A₂₈₀ OD reading was used to determine the concentration of the goat anti-mouse IgG and the A₆₅₀ OD reading was used to determine the degree of labeling based on the formula:

Moles dye per mole protein=A650× dilution factor/239,000× protein concentration (M)

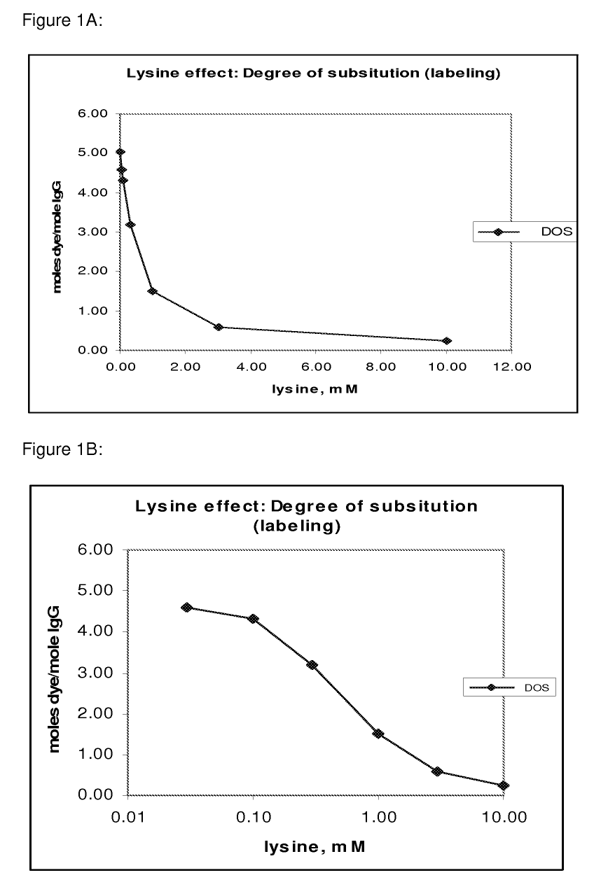

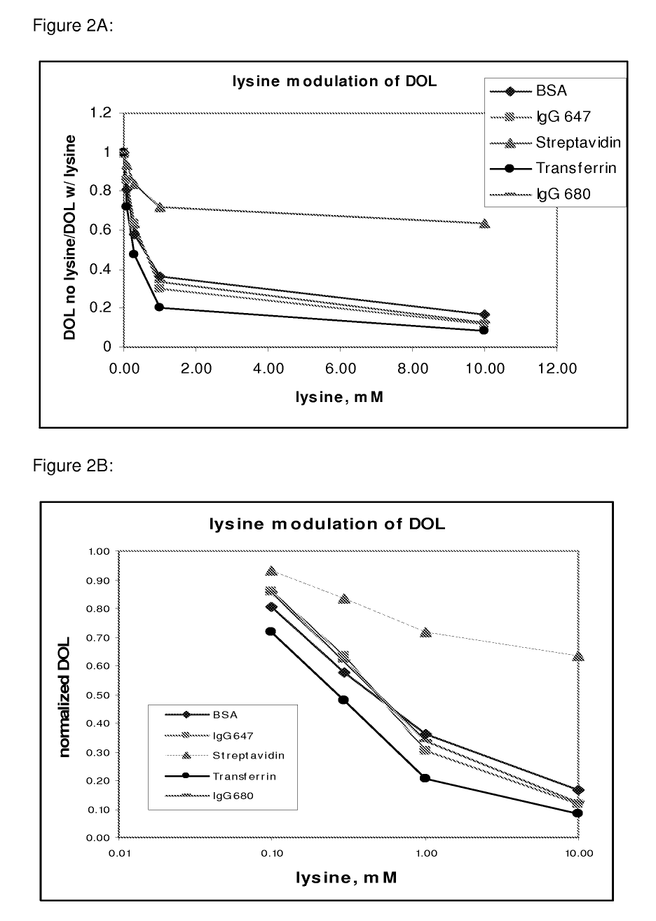

The influence on the degree of labeling is demonstrated in FIG. 1 where higher concentrations of lysine reduced the degree of labeling of the dye on the IgG in a somewhat linear fashion.

### Example 2

Use of lysine, a primary amine containing compound, as a reactive label competitor to control the DOL of Alexa Fluor® 647 and 680 dye conjugated to a Goat anti-mouse IgG, Bovine Serum Albumin (BSA), Streptavidin and Holotransferrin.

The IgG (Fortan Bioscience, Inc C-301-C-ABS lot 152-101-122004), Streptavidin (Prozyme), and Holotransferrin (SIGMA T4132-1G lot 035K0825) were prepares as 1 mg/ml stock solutions in PBS. The conjugation reactions were performed as described in Example 1 using Alexa Fluor 647 dye (Invitrogen Corp. A20186) and Alexa Fluor 680 (Invitrogen Corp. A20172) with lysine at a concentration of 0, 0.1, 0.3, 1.0, 3.0 mM.

The labeled proteins were purified and a DOL determined as described above. The change in the degree of labeling for these various proteins and for the previously obtained data above was normalized for each protein and dye by dividing the obtained degree of labeling at any lysine concentration by the degree of labeling with no lysine added. Result is expressed as a decimal fraction, e.g., 0.5 for reduction of label from 6 (no lysine) to 3 (with lysine) dye molecules per IgG. See, FIG. 2.

Example 1 and Example 2 demonstrates that free lysine can control DOL in a predictable way for any given protein/dye condition tested. When labeled with Alexa Fluor 647 dye BSA and IgG are nearly congruent, and transferrin appears slightly more sensitive to lysine-reduction of DOL. Stretpavidin appears to be less sensitive to the use of lysine as a reactive label competitor. The advantage and utility of this method is that predictable labeling modulation can be done without significantly changing the protein or dye concentration. This has many import aspects including controlling the quenching affects of too many dyes per protein and if the degree of labeling affects the pharmacokinetics of proteins, facile control of labeling would be very useful.

### Example 3

Effect of lysine concentration, incubation times, incubation temperatures and different proteins on the DOL with Alexa Fluor 647 dye or Alexa Fluor 680 dye (containing succinimidyl ester (SE) as the reactive group)

The degree of inhibition and variability with 60 minute (room temperature) compared to about 20 hours (on ice) incubation times was evaluated by performing a labeling reaction as described above based on a protein concentration of 1 mg/ml in PBS. Goat anti-rabbit IgG was labeled with Alexa Fluor 647 dye and Alexa Fluor 680 dye in the presence of 0, 0.3 mM and 1 mM concentration of free lysine.

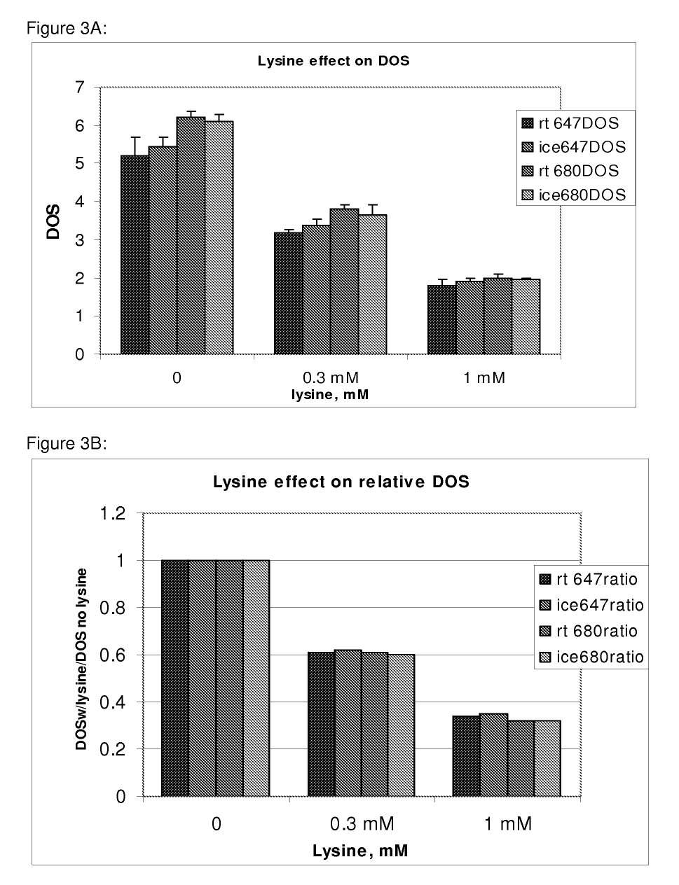

The protein concentration of the labeled antibody and the DOL was determined as described above. With the exception that antibody labeled with Alexa Fluor 680 dye had the absorbance read at A₆₇₉. See, FIG. 3.

The degree of inhibition and variability was evaluated by performing a labeling reaction as described above based on a protein concentration of 1 mg/ml in PBS or water. The proteins to be labeled were F(ab′)2 Goat anti-mouse (GAM) IgG (ZYMED 62-6300, lot 50594901, 2×1 mg, lyophilized); Fab′ Goat anti-rabbit (GAR) IgG Fc (Fortron Biosciences of Morrisville, N.C.); and holo-transferrin. The proteins were labeled with Alexa Fluor 647 dye and Alexa Fluor 680 dye in the presence of 0, 0.3 mM and 1 mM concentration of free lysine.

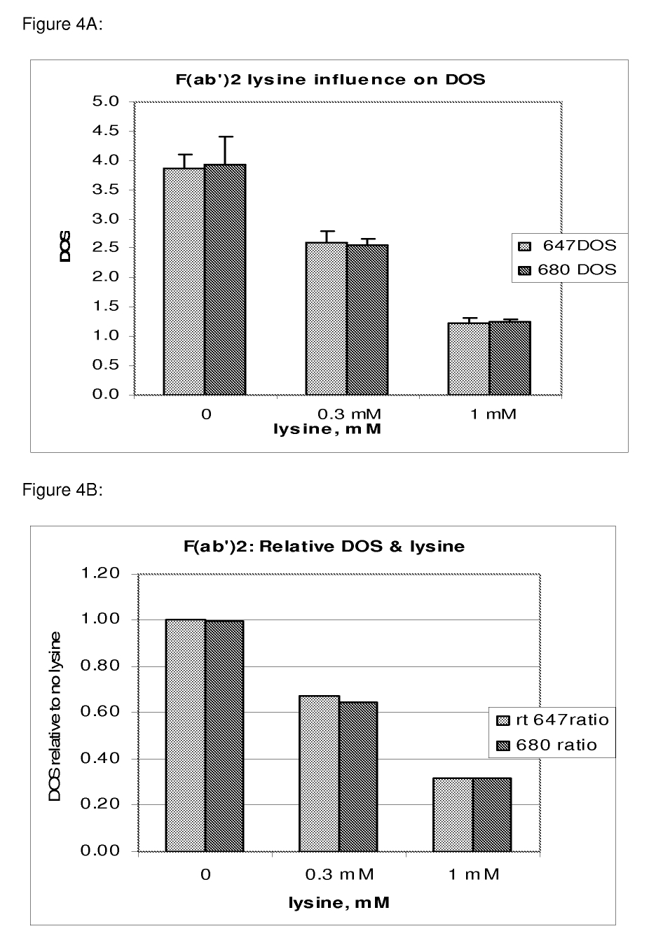

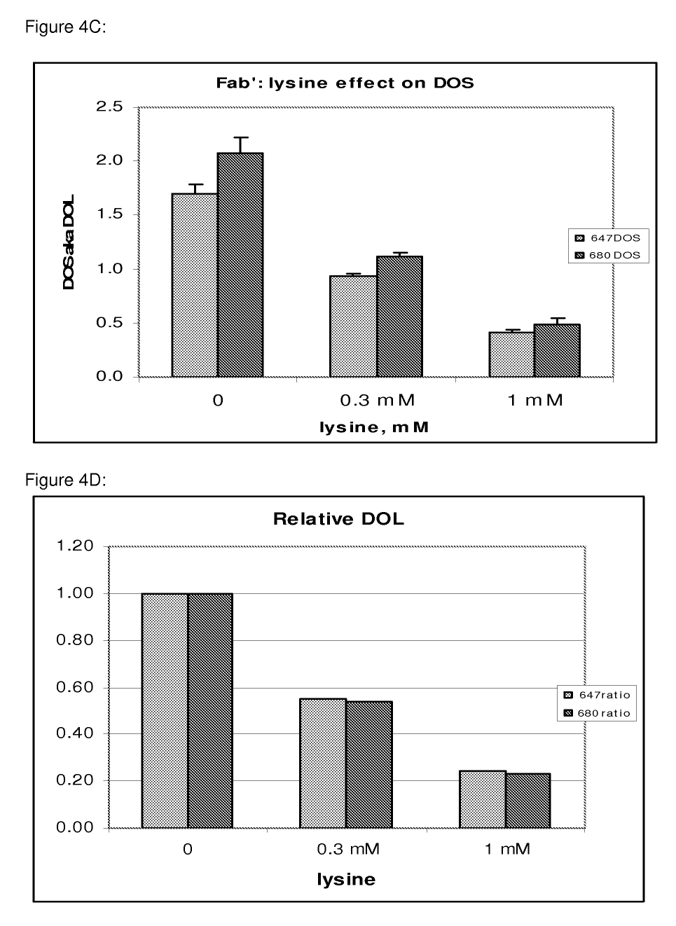

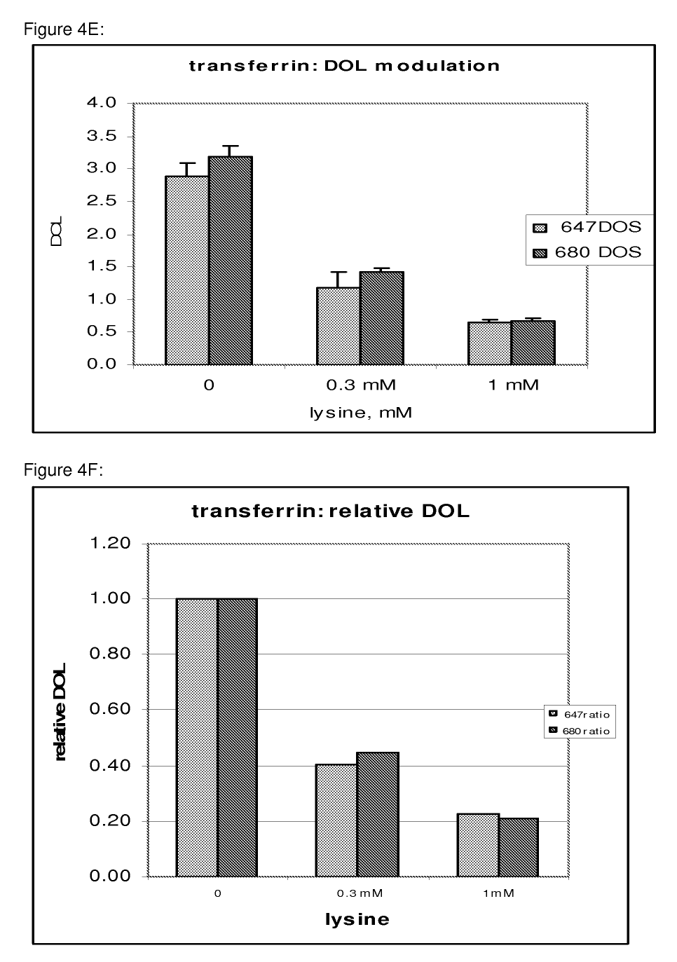

The protein concentration of the labeled proteins and the DOL was determined as described above. See, FIG. 4 and Table 3.

TABLE 3

| | | Dye | | | |
|---|---|---|---|---|---|
| | | (Alexa | | | |
| | Lysine, | Fluor) | Avg. | | Avg. |
| Sample | mM | SE | DOL +/− SD | Ratio | % yield |
| GAR 1 mg/ml | 0 | AF 647 | 5.2 +/− 0.5 | 1.00 | 71 +/− 2.8 |
| RT, 60 min | 0.3 | | 3.2 +/− 0.1 | 0.61 | 73 +/− 6.1 |
| | 1.0 | | 1.8 +/− 0.2 | 0.34 | 64 +/− 9.5 |
| GAR 1 mg/mL | 0 | | 5.5 +/− 0.2 | 1.00 | 68 +/− 2.4 |
| ice, 19.5 hr. | 0.3 | | 3.4 +/− 0.2 | 0.62 | 68 +/− 2.5 |
| | 1.0 | | 1.9 +/− 0.1 | 0.35 | 62 +/− 8.3 |
| GAR 1 mg/ml | 0 | AF 680 | 6.2 +/− 0.2 | 1.00 | 67 +/− 3.2 |
| RT, 60 min | 0.3 | | 3.8 +/− 0.1 | 0.61 | 64 +/− 1.6 |
| | 1.0 | | 2.0 +/− 0.1 | 0.32 | 63 +/− 3.9 |
| GAR 1 mg/mL | 0 | | 6.1 +/− 0.2 | 1.00 | 68 +/− 1.0 |
| ice, 19.5 hr. | 0.3 | | 3.7 +/− 0.3 | 0.60 | 66 +/− 1.3 |
| | 1.0 | | 2.0 +/− 0.0 | 0.32 | 60 +/− 0.6 |
| GAM IgG F(ab′)₂ | 0 | AF 647 | 3.9 +/− 0.2 | 1.00 | 46 +/− 2.4 |
| (0.6 mg/ml) | 0.3 | | 2.6 +/− 0.2 | 0.67 | 46 +/− 4.2 |
| (Zymed) RT, | 1.0 | | 1.2 +/− 0.1 | 0.32 | 45 +/− 6.9 |
| 60 min | 0 | AF 680 | 3.9 +/− 0.5 | 1.00 | 60 +/− 5.5 |
| | 0.3 | | 2.6 +/− 0.1 | 0.67 | 45 +/− 5.5 |
| | 1.0 | | 1.3 +/− 0.0 | 0.32 | 51 +/− 3.5 |
| GAR IgG Fab′ | 0 | AF 647 | 1.7 +/− 0.1 | 1.00 | 68 +/− 3.1 |
| 1 mg/ml, RT, | 0.3 | | 0.9 +/− 0.0 | 0.55 | 69 +/− 2.9 |
| 60 min | 1.0 | | 0.4 +/− 0.0 | 0.24 | 64 +/− 1.1 |
| | 0 | AF 680 | 2.1 +/− 0.2 | 1.00 | 64 +/− 4.1 |
| | 0.3 | | 1.1 +/− 0.0 | 0.54 | 66 +/− 1.9 |
| | 1.0 | | 0.5 +/− 0.1 | 0.23 | 63 +/− 1.1 |
| holo-transferrin | 0 | AF 647 | 2.9 +/− 0.2 | 1.00 | 75 +/− 0.7 |
| 1 mg/ml, RT, | 0.3 | | 1.2 +/− 0.2 | 0.40 | 77 +/− 2.6 |
| 60 min | 1.0 | | 0.7 +/− 0.0 | 0.23 | 72 +/− 5.1 |
| | 0 | AF 680 | 3.2 +/− 0.2 | 1.00 | 77 +/− 4.1 |
| | 0.3 | | 1.4 +/− 0.1 | 0.45 | 75 +/− 2.2 |
| | 1.0 | | 0.7 +/− 0.0 | 0.21 | 68 +/− 6.2 |

These results demonstrate that the addition of lysine consistently and reproducibly alters the DOL of reactive dye conjugated to different proteins. The degree of lysine modulation of IgG and (Fab′)₂ labeling is similar. The labeling of transferrin is more strongly inhibited by lysine, possibly due to the different amino acid composition. The relative DOL of Fab′, (Fab′)₂, and IgG is roughly proportional to their respective molecular weights. Standard deviations indicate that for each protein and each condition labeling is consistent. Yields are variable, but for IgG's tend to be 65 to 70%.

The effects of protein concentration on protein DOL was evaluated by performing a labeling reaction as described above based on protein concentration of 3 mg/ml, 1 mg/ml and 0.3 mg/ml in PBS. Goat anti-rabbit IgG was labeled with Alexa Fluor 647 dye and Alexa Fluor 680 dye in the presence of 0, 0.3 mM and 1 mM concentration of free lysine.

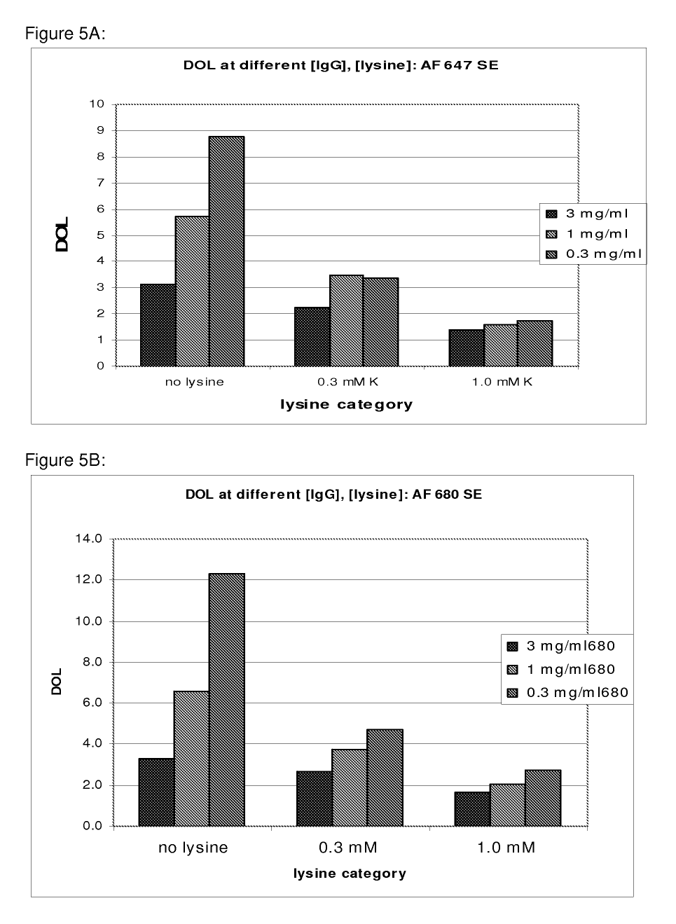

The protein concentration of the labeled antibody and the DOL was determined as described above. With the exception that antibody labeled with Alexa Fluor 680 dye had the absorbance read at A₆₇₉. See, FIG. 5.

TABLE 4

| | Lysine, | Dye (Alexa | | | |
|---|---|---|---|---|---|
| Sample | mM | Fluor) SE | DOL | Ratio | % yield |
| GAR 3 mg/ml | 0 | AF 647 | 3.1 | 1.00 | Not Done |
| | 0.3 | | 2.2 | 0.72 | N D |
| | 1.0 | | 1.4 | 0.45 | N D |
| GAR 1 mg/ml | 0 | | 5.7 | 1.00 | N D |
| | 0.3 | | 3.5 | 0.61 | N D |
| | 1.0 | | 1.6 | 0.28 | N D |
| GAR 0.3 mg/mL | 0 | | 8.8 | 1.00 | N D |
| | 0.3 | | 3.4 | 0.38 | N D |
| | 1.0 | | 1.7 | 0.20 | N D |
| GAR 3 mg/ml | 0 | AF 680 | 3.3 | 1.00 | 75 |
| | 0.3 | | 2.7 | 0.82 | 76 |
| | 1.0 | | 1.6 | 0.50 | 67 |
| GAR 1 mg/ml | 0 | | 6.6 | 1.00 | 66 |
| | 0.3 | | 3.7 | 0.57 | 68 |
| | 1.0 | | 2.0 | 0.31 | 60 |
| GAR 0.3 mg/mL | 0 | | 12.3 | 1.00 | 38 |
| | 0.3 | | 4.7 | 0.38 | 46 |
| | 1.0 | | 2.7 | 0.22 | 39 |

The effects of protein concentration on protein DOL and lysine modulation of protein DOL reflect the relationship between molar ratio of protein to dye-SE and the competition between lysine and protein for the dye-SE substrate. Yields for antibody concentration of 0.3 mg/ml are relatively low.

### Example 4

Effect of lysine concentration, incubation times, and incubation temperatures on the DOL with Alexa Fluor 750 dye (containing succinimidyl ester (SE) as the reactive group) conjugated to Goat anti-rabbit IgG.

The degree of inhibition and variability with 60 minute (room temperature) compared to about 20 hours (on ice) incubation times was evaluated by performing a labeling reaction as described above based on a protein concentration of 1 mg/ml in PBS and a DOL range of 2 to 4 dyes per protein. Goat anti-rabbit IgG was labeled with Alexa Fluor 750 dye in the presence of 0, 0.3 mM and 1 mM concentration of free lysine.

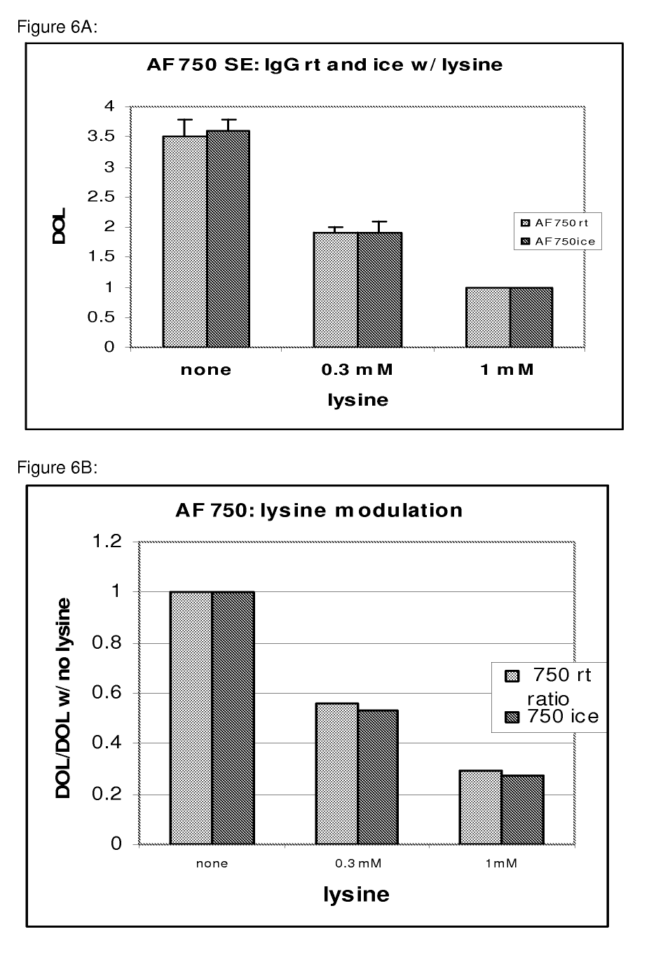

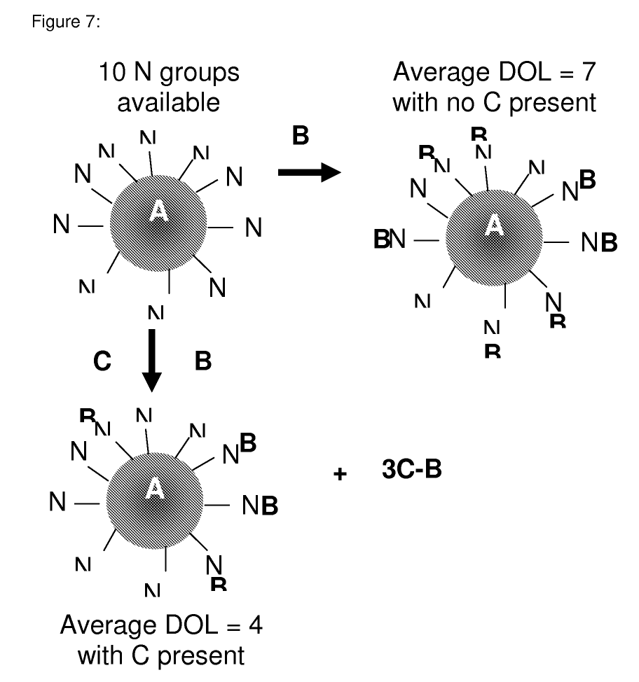

The protein concentration of the labeled antibody and the DOL was determined as described above, with the exception that antibody labeled with Alexa Fluor 750 dye had the absorbance read at A₇₅₀. See, FIG. 6.

TABLE 5

| | | Dye | | | |
|---|---|---|---|---|---|
| | | (Alexa | | | |
| | Lysine, | Fluor) | Avg. | | Avg. |
| Sample | mM | SE | DOL +/− SD | Ratio | % yield |
| GAR RT, 60 min | 0 | AF 750 | 3.5 +/− 1.0 | 1.00 | 72 +/− 2.3 |
| 1 mg/ml | 0.3 | | 1.9 +/− 0.1 | 0.56 | 69 +/− 2.4 |
| | 1.0 | | 1.0 +/− 0.0 | 0.29 | 66 +/− 4.3 |
| GAR ice, 20 hr | 0 | | 3.6 +/− 0.2 | 1.00 | 74 +/− 1.2 |
| 1 mg/ml | 0.3 | | 1.9 +/− 0.2 | 0.56 | 71 +/− 5.0 |
| | 1.0 | | 1.0 +/− 0.0 | 0.29 | 63 +/− 4.6 |

Lysine modulation with Alexa Fluor 750 dye was comparable to the results obtained with Alexa Fluor 647 dye and Alexa Fluor 680 dye, See Example 1-3. To achieve the relatively lower DOL (2-4 vs. 3-6) with this dye, the molar ratio of the dye: protein is less than that with the Alexa Fluor 647 dye and Alexa Fluor 680 dye, but is sufficiently high that the effect of lysine at 0.3 and 1.0 mM allows useful modulation of DOL.

### Example 5

Conjugation reactions are performed at room temperature (18-26° C.). All solutions, including the antibody solution, are equilibrated to room temperature. The antibody solution is free of ammonium ions, primary amines or contaminating polypeptides and proteins. If the antibody is in or has been lyophilized from an unsuitable buffer (such as Tris or glycine) or purified with ammonium sulfate the buffer is replaced with 1× phosphate buffered saline (PBS) by dialysis or gel filtration. The presence of low concentration of sodium azide (≦3 mM) or thimerosal does not interfere with the conjugation reaction.

A solution of sodium bicarbonate and PBS is dissolved completely by vortexing or repeated pipetting. A solution containing lysing and 500 μl antibody solution (1 mg/ml to 10 mg/ml) is added to the sodium bicarbonate/PBS solution. The mixture is transferred to a reaction vial containing lyophilized reactive dye. The antibody/dye solution is incubated for 60 minutes at room temperature and protected from light.

The sample is then loaded onto a column, allowing the reaction mixture to absorb into the column bed. The column is washed with 1.4 mL of PBS. The antibody-dye conjugate is eluted by applying ˜1 mL PBS to the column and collecting the eluate in a 1.5 micro-centrifuge tube or appropriate equivalent. The dye-conjugated protein is a light-to-medium blue liquid (Alexa Fluor 680 conjugates) or blue-green liquid (Alexa Fluor 750 conjugates). The unincorporated dye will remain on the column as a broad, intense band.

The dye-conjugate eluate is then sterile-filtered by fitting a 1 mL syringe into the sterile filter disc, removing the plunger, and pipetting the dye-antibody eluate into the syringe. The plunder is then replaced and eluate filtered into an appropriate sterile tube with one, smooth movement.

The peak absorbances of the purified conjugate are determined by diluting a sample of the purified conjugate with PBS 1:10 or 1:20 and measuring the protein absorbance at 280 nm and dye at 679 nm (Alexa Fluor 680 conjugates) or 750 nm (Alexa Fluor 750) conjugates or peak protein and dye absorbances are determined by scanning absorbance.

The DOL is determined for particular dyes by the following equations:

Alexa Fluor 680 conjugates

Protein concentration (M): [A₂₈₀-(A₆₇₉×0.05)]× dilution factor/203,000

Moles of dye/mole of protein (DOL):

A₆₇₉× dilution factor/(184,000× protein concentration (M))

For Alexa Fluor 750 conjugates:

Protein concentration (M): [A₂₈₀-(A₇₅₀×0.034)]× dilution factor/203,000

Moles of dye/mole of protein (DOL):

A₇₅₀× dilution factor/(270,000× protein concentration (M))

The above allows for simple conjugation and purification protocols and is optimized for in vivo imaging. The reactions produce antibody-fluorophore conjugates that are immediately suitable for animal use: azide free, sterile filtered. The reaction labels antibodies at DOL of 1.75-2.75 over a 10-fold protein concentration range with no adjustments in reaction volume, dye concentration, or antibody concentration necessary. No additional post-label reactions are required. Fluorescent conjugate purification is with a rapid, simple gravity column protocol that is complete within 5-10 minutes, with excellent reproducibility. No spin column is required.

All publications referred to within this document are incorporated by reference to the same extent as if each individual publication was specifically and individually indicated to be incorporated by reference.

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

We claim:

1. A method for controlling the degree of labeling (DOL) of a carrier molecule or solid support, wherein the method comprises:
a) contacting the carrier molecule or solid support with a reactive label to form a labeling solution;
b) contacting the labeling solution with a reactive label competitor to form a controlled labeling solution; and
c) incubating the controlled labeling solution for an appropriate amount of time whereby the degree of labeling of the carrier molecule or solid support is controlled.

2. The method according to claim 1, wherein the carrier molecule comprises a amino acid, a peptide, a protein, a polysaccharide, a nucleotide, a nucleoside, an oligonucleotide, a nucleic acid, a hapten, a psoralen, a drug, a hormone, a lipid, a lipid assembly, a synthetic polymer, a polymeric microparticle, a biological cell or a virus.

3. The method according to claim 1, wherein the carrier molecule comprises an antibody or fragment thereof, an avidin or streptavidin, a biotin, a blood component protein, a dextran, an enzyme, an enzyme inhibitor, a hormone, an IgG binding protein, a fluorescent protein, a growth factor, a lectin, a lipopolysaccharide, a microorganism, a metal binding protein, a metal chelating moiety, a non-biological microparticle, a peptide toxin, a phosphotidylserine-binding protein, a structural protein, a small-molecule drug, or a tyramide.

4. The method according to claim 1, wherein the solid support comprises a microfluidic chip, a silicon chip, a microscope slide, a microplate well, silica gels, polymeric membranes, particles, derivatized plastic films, glass beads, cotton, plastic beads, alumina gels, polysaccharides, polyvinylchloride, polypropylene, polyethylene, nylon, latex bead, magnetic bead, paramagnetic bead, and superparamagnetic bead.

5. The compound according to claim 1, wherein the solid support comprises Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose and starch.

6. The method according to claim 1, wherein the reactive label comprises a fluorophore, a phosphorescent dye, a tandem dye, a particle, an electron transfer agent, biotin or a radioisotope.

7. The method according to claim 6, wherein the fluorophore is dansyl, xanthene, naphthalene, borapolyazaindacene, coumarin, cyanine, pyrene, or derivatives thereof.

8. The method according to claim 6, wherein the fluorophore has an emission spectra greater than about 600 nm.

9. The method according to claim 6, wherein the fluorophore has an emission spectra greater than about 620 nm.

10. The method according to claim 6, wherein the fluorophore has an emission spectra greater than about 650 nm.

11. The method according to claim 6, wherein the fluorophore has an emission spectra great than about 700 nm.

12. The method according to claim 6, wherein the fluorophore has an emission spectra greater than about 750 nm.

13. The method according to claim 6, wherein the fluorophore has an emission spectra greater than about 800 nm.

14. The method according to claim 6, wherein the particle label comprises a nanocrystal or a resonance light scattering particle.

15. The method according to claim 1, wherein the reactive label comprises a reactive group.

16. The method according to claim 15, wherein the reactive group comprises an acrylamide, an activated ester of a carboxylic acid, a carboxylic ester, an acyl azide, an acyl nitrile, an aldehyde, an alkyl halide, an anhydride, an aniline, an amine, an aryl halide, an azide, an aziridine, a boronate, a diazoalkane, a haloacetamide, a haloalkyl, a halotriazine, a hydrazine, an imido ester, an isocyanate, an isothiocyanate, a maleimide, a phosphoramidite, a reactive platinum complex, a silyl halide, a sulfonyl halide, a silanol, or a thiol.

17. The compound according to claim 15, wherein the reactive group comprises a carboxylic acid, succinimidyl ester of a carboxylic acid, hydrazide, amine and a maleimide.

18. The method according to claim 1, wherein the reactive label competitor comprises an amino or thiol group.

19. The method according to claim 1, wherein the reactive label comprises α-amino acids, β-amino acids, amino alcohols, ε-amino acids, primary amine containing compounds or reactive secondary amine-containing compounds.

20. The method according to claim 1, wherein the reactive label competitor comprises D-lysine, L-lysine, D,L-lysine, ethanolamine, 5-amino caproic acid, or ammonia (NH₃).

21. The method according to claim 1, wherein the reactive label competitor is L-Lysine Hydrochloride.

22. The method according to claim 1, wherein the reactive label competitor comprises α-mercapto acids, β-mercapto acids, mercapto alcohols, ε-mercapto acids, primary mercaptan compounds or reactive secondary mercaptan compounds.

23. The method according to claim 1, wherein the reactive label competitor comprises D-cysteine, L-cysteine, D,L-cysteine, mercaptoethanol, 5-mercapto caproic acid, or H₂S.

24. The method according to claim 20, wherein the DOL is about 4 when the concentration of lysine is about 0.3 mM.

25. A method of modulating the amount of reactive label present in a solution, said method comprising:
a) contacting a solution comprising a carrier molecule or solid support with a reactive label to form a labeled carrier molecule or labeled solid support; and
b) contacting the solution with a reactive label competitor to form a labeled competitor;
wherein the amount of reactive label in the solution is attenuated or eliminated after contacting the reactive label with the reactive label competitor.

26. The method of claim 25, further comprising a step of separating labeled competitor from the labeled carrier molecule or labeled solid support.

27. The method of claim 25, wherein the amount of labeled carrier molecule or labeled solid support is essentially unaffected by the concentration of carrier molecule or solid support in solution.

28. The method of claim 25, wherein the pH of the solution is between 3 and 10.

29. The method of claim 25, wherein the pH of the solution is between 7 and 9.

30. The method according to claim 1, further comprising a buffer.

31. The method according to claim 25, further comprising a buffer.

32. A method for monitoring the degree of labeling (DOL) of a carrier molecule or solid support, said method comprising:
a) contacting a solution comprising a carrier molecule or solid support with a reactive label to form a labeled carrier molecule or labeled solid support; and
b) contacting the solution with a reactive label competitor to form a labeled competitor, wherein the reactive label competitor quenches or is capable of FRET interaction with the reactive label;
wherein the degree of labeling (DOL) is monitored by the amount of quenching or FRET by the reactive label competitor.

33. A kit for controlling the degree of labeling (DOL) of a carrier molecule or solid support, wherein the kit comprises:
a) carrier molecule or solid support;
b) a reactive label;
c) a reactive label competitor; and
d) instructions for performing a method resulting in the controlled degree of labeling of the carrier molecule or solid support.

34. The kit of claim 33, further comprising a buffer.
